(19) United States
(12) Patent Application Publication
(10) Pub. No.: US 2026/0108541 A1
(43) Pub. Date: Apr. 23, 2026

(54) AMINERGIC PHYTOCANNABINOIDS DERIVATIVES AS MULTI-TARGET AGENTS FOR NEUROLOGICAL DISORDERS

(71) Applicant: University of South Carolina, Columbia, SC (US)

(72) Inventors: Juan Francisco Leon Oyola, West Columbia, SC (US); Zachary I. Stryker, Columbia, SC (US)

(73) Assignee: University of South Carolina, Columbia, SC (US)

(21) Appl. No.: 19/365,329

(22) Filed: Oct. 22, 2025

Related U.S. Application Data
(60) Provisional application No. 63/710,160, filed on Oct. 22, 2024.

(51) Int. Cl. A61K 31/00; A61P 25/00; C07C 217/74

(52) U.S. Cl. CPC A61K 31/658; A61P 25/00; C07C 217/74

(57) ABSTRACT

Compounds, methods of making the compounds, and methods of using the compounds to treat neurological disorders in mammals, including humans. The compounds are aminergic phytocannabinoid derivatives, including cannabinoid (CBD) derivatives functionalized at position 7 of the CBD molecule and tetrahydrocannabinol (THC) derivatives functionalized at position 11 of the THC molecule.

### CROSS-REFERENCE TO RELATED APPLICATIONS

Priority is hereby claimed to provisional application Ser. No. 63/710,160, filed Oct. 22, 2024, which is incorporated herein by reference.

### FEDERAL FUNDING STATEMENT

This invention was made with government support under P20 GM109091 awarded by the National Institutes of Health. The government has certain rights in the invention.

### BACKGROUND

Neurological disorders, such as epilepsy, multiple sclerosis, Alzheimer's disease, and Parkinson's disease, represent a significant burden on public health worldwide. These disorders are often multifactorial, involving complex interactions between neurotransmitter systems, inflammatory pathways, oxidative stress, and neurodegeneration. Depressive disorders, such as major depressive disorder and dysthymia, have been linked to neurotransmitter imbalances, neuroinflammation, and changes in brain structure and function. These disorders involve not only psychological factors but also neurological processes that affect mood regulation, neuroplasticity, and stress response systems.

The therapeutic potential of phytocannabinoids, a class of naturally occurring compounds derived from Cannabis sativa, has gained significant attention in recent years due to their therapeutic potential. More than 113 different cannabinoids have been isolated from C. sativa and are classified into distinct types: cannabigerols (CBGs), cannabichromenes (CBCs), cannabidiols (CBDs), (−)-Δ⁹-trans-tetrahydrocannabinols (Δ⁹-THCs), (−)-Δ⁸-trans-tetrahydrocannabinols (Δ⁸-THCs), cannabicyclols (CBLs), cannabielsoins (CBEs), cannabinols (CBNs), cannabinodiols (CBNDs), cannabitriols (CBTs), and the miscellaneous cannabinoids (ElSohly and Slade, “Chemical constituents of marijuana: The complex mixture of natural cannabinoids,” Life Sci. 2005, 78(5): 539-548; Gülck and Møller, “Phytocannabinoids: Origins and Biosynthesis,” Trends Plant Sci. 2020, 25(10): 985-1004).

CBD is a non-psychoactive cannabinoid known for its anti-inflammatory, anti-anxiety, and anti-seizure effects. It has shown promise in the treatment of conditions such as epilepsy, neurodegenerative disorders, and anxiety-related symptoms. See Hampson et al. “Cannabidiol and (−)Delta9-tetrahydrocannabinol are neuroprotective antioxidants,” Proc. Natl. Acad. Sci. U.S.A. 1998, 95: 8268; Zuardi et al. “Action of cannabidiol on the anxiety and other effects produced by delta 9-THC in normal subjects,” Psychopharmacology—Ber. 1982, 76: 245; Cunha et al. “Chronic administration of cannabidiol to healthy volunteers and epileptic patients,” Pharmacol. 1980, 21: 175; Consroe. “Brain cannabinoid systems as targets for the therapy of neurological disorders,” Neurobiol. Disease 1998, 5: 534.

Δ⁸-THC, a structural analog of the more commonly known Δ⁹-THC, has a milder psychoactive effect and is being explored for its neuroprotective and anxiolytic properties (Kruger and Kruger. “Delta-8-THC: Delta-9-THC's nicer younger sibling?” J Cannabis Res. 2022, 4: 4). Its reduced psychoactivity, compared to Δ⁹-THC, makes it an attractive candidate for therapeutic applications where the psychoactive side effects of Δ⁹-THC are undesirable.

In addition to the phytocannabinoids themselves, their various derivatives with better pharmacological activity or overcoming some defects in use have attracted more attention from researchers. For example, terpene hydrocannabinol and terpene-modified derivatives such as H₂CBD are more active than CBD and do not convert to THC to cause side effects (Mascal et al. “Synthetic, non-intoxicating 8,9-dihydrocannabidiol for the mitigation of seizures,” Sci. Rep. 2019, 9: 7778). The fluorinated derivative 4′-F-CBD shows higher potency than CBD in behavioral assays in mice predictive of anxiolytic, antidepressant, antipsychotic and anti-compulsive activity (Breuer et al. “Fluorinated Cannabidiol Derivatives: Enhancement of Activity in Mice Models Predictive of Anxiolytic, Antidepressant and Antipsychotic Effects,” PLoS One. 2016; 11(7): e0158779). CBDV (cannabidivarin), a homolog of CBD having a shorter side chain, has been studied for its potential in treating epilepsy and other neurological disorders (Hurley et al. “Efficacy and safety of cannabidivarin treatment of epilepsy in girls with Rett syndrome: A phase 1 clinical trial,” Epilepsia. 2022. 63: 1736-1747).

In recent years, the concept of the “magic shotgun” has gained attention in the pharmaceutical field. Unlike the traditional “magic bullet” approach, which seeks to identify a single compound that selectively targets one biological pathway, the “magic shotgun” strategy recognizes that many diseases, particularly complex ones like neurological and depressive disorders, require interventions that simultaneously modulate multiple targets. The most successful antipsychotic drugs may interact with multiple targets in the brain to attenuate different pathways in the central nervous system simultaneously. See e.g., Roth et al. “Magic shotguns versus magic bullets: selectively non-selective drugs for mood disorders and schizophrenia,” Nat. Rev. Drug Discov. 2004, 3: 353-359.

The present disclosure provides novel phytocannabinoid derivatives as multi-target agents, offering new therapeutic strategies for the treatment and management of neurological disorders.

### SUMMARY

Provided herein are aminergic phytocannabinoid derivatives, methods for their production, and their pharmaceutical applications. The aminergic phytocannabinoid derivatives include cannabinoid (CBD) derivatives functionalized at position 7 of the CBD molecule and tetrahydrocannabinol (THC) derivatives functionalized at position 11 of the THC molecule. CBD from natural cannabis extracts is transformed through a sequence of four reactions to produce a dimethyl ether counterpart that has a chlorine substitute at position 7 of the molecule (compound 5 of Scheme 1). In a similar pathway, THC is converted to a methyl ether counterpart having a chlorine substitute at position 11 of the molecule (e.g., compound Δ⁸-5 of Scheme 2). The compounds are subsequently used as the starting material to introduce nitrogen- or oxygen-containing functionalities. The CBD and THC derivatives having novel chemical structures can be used to treat a variety of health conditions, including, but not limited to, neurodegenerative disorders, valvular heart disease, and/or mood disorders, by acting as “magic shotguns”, i.e., through a range of highly promiscuous interactions at varying targets. They may act in the brain as anti-depressants, anxiolytics, or anti-psychotics through regulation of central serotonin and dopamine receptors and may act peripherally to prevent pain or the onset of valvular heart conditions through opioid receptors and serotonin receptors, respectively.

Specifically, disclosed and claimed herein is a compound of Formula I or II:

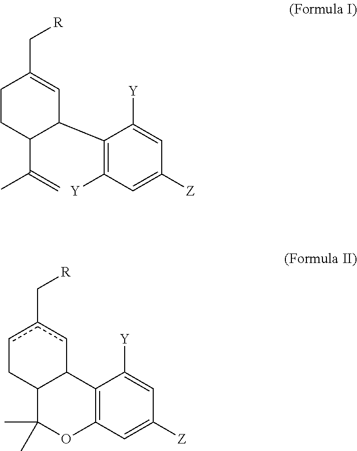

wherein each --- between adjacent atoms in Formula II represents a bond that is present or absent, with the proviso that one bond is present and another is absent;
Y in each Formula is independently selected from the group consisting of hydroxy, alkoxy, acyloxy, and —COOR⁰, wherein R⁰ is selected from the group consisting of unsubstituted or substituted C₁-C₁₂ alkyl, C₂-C₁₂ alkenyl, C₂-C₁₂ alkynyl, and alkoxyalkyl;
Z in each Formula is independently selected from the group consisting of C₁-C₁₂ alkyl; and
each R is independently selected from the group consisting of:

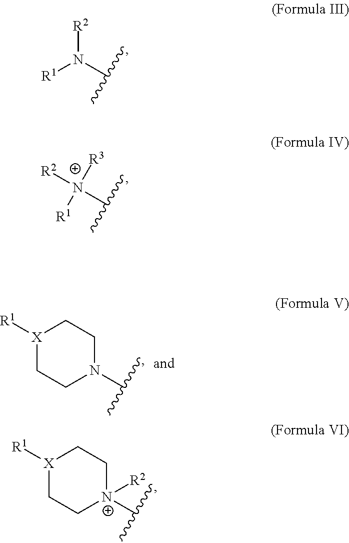

wherein R¹, R², and R³, if present, are each independently selected from the group consisting of hydrogen, unsubstituted or substituted C₁-C₁₂ alkyl, C₂-C₁₂ alkenyl, C₂-C₁₂ alkynyl, aryl, arylalkyl, and heterocyclyl; and
X is selected from the group consisting of carbon, nitrogen, and oxygen.

In one version, the compound is a derivative of CBD, having a structure of Formula I. Y maybe methoxy (—O—CH₃), hydroxy (—OH), or acetoxy (—O—C(═O)—CH₃).

In another version, the compound is a derivative of THC (Δ⁸-THC or Δ⁹-THC), having a structure of Formula II. Y maybe methoxy (—O—CH₃), hydroxy (—OH), or acetoxy (—O—C(═O)—CH₃).

In one version, R is Formula III, wherein R¹ and R² are each independently selected from the group consisting of hydrogen, unsubstituted or substituted C₁-C₁₂ alkyl, and arylalkyl. Exemplary substituents for R¹ and R² include, but are not limited to, hydrogen, methyl, ethyl, isopropyl, and diphenylmethyl.

In another version, R is Formula IV, wherein R¹, R², and R³ are each independently selected from the group consisting of unsubstituted or substituted C₁-C₁₂ alkyl. Exemplary substituents for R¹, R², and R³ include, but are not limited to, methyl, ethyl, and isopropyl.

In another version, R is Formula V, wherein R¹ is selected from the group consisting of unsubstituted or substituted C₁-C₁₂ alkyl, aryl, arylalkyl, and heterocyclyl. In certain embodiments, the arylalkyl is a halogen substituted arylalkyl. Exemplary substituents for R¹ include, but are not limited to, methyl, phenyl, pyrrolidinyl (—NC₄H₈), and halogen substituted phenylmethyl (e.g., —CH₂(p-C₆H₄F) or —CH₂(p-C₆H₄Cl).

In another version, R is Formula VI, wherein R¹ and R² are each independently selected from the group consisting of hydrogen, unsubstituted or substituted C₁-C₁₂ alkyl, and arylalkyl. In some embodiments, the arylalkyl is a halogen substituted arylalkyl. Exemplary substituents for R¹ and R² include, but are not limited to, hydrogen, methyl, and halogen substituted phenylmethyl (e.g., —CH₂(p-C₆H₄F) or —CH₂(p-C₆H₄Cl).

Also disclosed and claimed herein is a pharmaceutical composition comprising the compound as disclosed herein, optionally in combination with a pharmaceutically suitable carrier.

Also disclosed and claimed herein is a method of treating neurological disorders in a mammal, comprising administering a therapeutically effective amount of the compound as disclosed herein. The mammal may be a human.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1 shows an exemplary synthetic pathway and exemplary functionalities of CBD derivatives.

FIGS. 2-10 show MS spectra of compounds 5 (5-Me; FIG. 2), 6d (6-IPA-Me; FIG. 3), 6e (6-DMA-Me; FIG. 4), 6f (6-DIPA-Me; FIG. 5), 6h (6-TMA-Me; FIG. 6), 6i (6-TEA-Me; FIG. 7), 7e (6-DMA-OH; FIG. 8), 7t (6-CBPz-OH; FIG. 9), and 8e (6-DMA-Ac; FIG. 10).

FIG. 11 binding affinity (Kᵢ) of compounds 6d (6-IPA-Me), 6e (6-DMA-Me), 6h (6-TMA-Me), and 6i (6-TEA-Me) against a panel of neurotransmitter receptors. Lower Kᵢ values indicate higher affinity of the compound for the receptor.

FIG. 12 shows secondary radioligand binding (i.e. dose-response) data for compounds 6d (6-IPA-Me), 6e (6-DMA-Me), 6h (6-TMA-Me), and 6i (6-TEA-Me) towards Sigma-1 or Sigma-2 receptors, compared to the sigma receptor ligand Haloperidol.

FIG. 13 shows whole-cell patch clamp experiment data for compound 6e (6-DMA-Me) in primary mouse hippocampal neurons. FIG. 13 displays the ability of compound 6e to potentiate NMDA receptor field excitatory post-synaptic potentials (fEPSPs), which can be reversed in the presence of a sigma-1 receptor antagonist, BD1047. (Panel A) Field excitatory post synaptic potentials were recorded from Schaffer collaterals. (Panel B) NMDA receptor potentials were isolated with AMPA receptor antagonist DNQX and could be blocked by application of the NMDA receptor antagonist APV (50 uM). (Panel C) Compound 6e potentiates NMDA receptor fEPSPs via the sigma-1 receptor (t(11)=2.312, p<0.05).

### DETAILED DESCRIPTION

### Abbreviations and Definitions

ACN=Acetonitrile; CBD=Cannabidiol; DCM=Dichloromethane; DIPA=Diisopropylamine; DMA=Dimethylamine; IPA=Isopropylamine; MS=Mass Spectrometry; MeOH=Methanol; MsCl=Methanesulfonyl chloride; NMR=Nuclear Magnetic Resonance; TEA=Triethylamine; THC=Tetrahydrocannabinol; TMA=Trimethylamine.

Cannabidiol (CBD) is a naturally occurring phytocannabinoid derived from the Cannabis sativa plant. It is a bicyclic compound with the molecular formula C₂₁H₃₀O₂, as shown below with carbon numbering. Unlike its counterpart Δ⁹-tetrahydrocannabinol (THC), CBD is non-psychoactive, meaning it does not produce the euphoric or intoxicating effects associated with THC. Cannabidiol interacts with various molecular targets, including cannabinoid receptors (CB1 and CB2), as well as non-cannabinoid receptors such as serotonin receptors (e.g., 5-HT1A) and transient receptor potential (TRP) channels. CBD as used herein can be isolated from cannabis extracts or synthesized chemically.

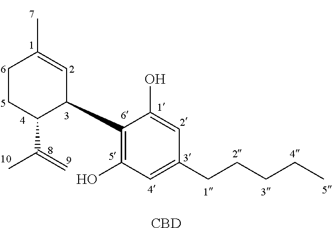

Tetrahydrocannabinol (THC) is another naturally occurring phytocannabinoid derived from the Cannabis sativa plant. It has the chemical formula C₂₁H₃₀O₂ and is characterized by its tetrahydrocannabinol structure. As used herein, THC encompasses all isomeric forms, including but not limited to Δ⁹-THC and Δ⁸-THC, which interact with cannabinoid receptors in the endocannabinoid system.

Δ⁹-THC is a primary psychoactive compound found in the Cannabis sativa plant. It has a structure as shown below with carbon numbering. It is known for its psychotropic effects, which include alterations in mood, perception, and cognitive function. It exerts its effects primarily through interaction with cannabinoid receptors CB1 and CB2 in the endocannabinoid system. As used herein, Δ⁹-THC can be isolated from cannabis extracts or synthesized chemically.

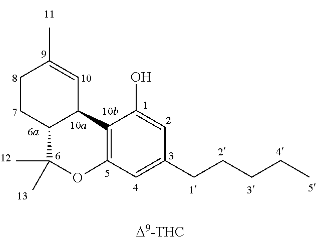

Δ⁸-THC is a less abundant cannabinoid in the cannabis plant, and it differs from Δ⁹-THC in its chemical structure. Δ⁸-THC has a double bond on the 8^{th} carbon chain, while Δ⁹-THC has a double bond on the 9^{th} carbon chain. The structure of Δ⁸-THC is shown below with carbon numbering. It exhibits psychoactive properties, though typically reported to be less potent than Δ⁹-THC. Δ⁸-THC interacts primarily with cannabinoid receptor CB1 in the endocannabinoid system, influencing mood, cognition, and perception. As used herein, Δ⁸-THC can be isolated from cannabis extracts or synthesized chemically.

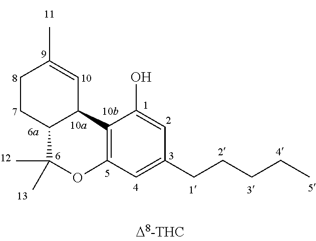

The term “acyloxy” refers to a substituent of the formula —O—C(═O)—R, where R represents a hydrocarbyl group, such as an alkyl, alkenyl, alkynyl, aryl, aralkyl, or heteroaryl group, optionally substituted. Examples of acyloxy groups include, without limitation, acetoxy (—O—C(═O)—CH₃), propionyloxy (—O—C(═O)—CH₂CH₃), and benzoyloxy (—O—C(═O)—C₆H₅).

The term “alkoxy” is an alkyl group singularly bonded to oxygen: —O—R, wherein R is an alkyl group. Examples include methoxy, ethoxy, etc.

The term “alkoxyalkyl” refers to a substituent in which an alkoxy group (—O—R) is bonded through an alkyl linker to the point of attachment. Suitable alkyl linkers may be linear, branched, or cyclic, and which may be unsubstituted or substituted. Non-limiting examples of alkoxyalkyl groups include methoxymethyl (—CH₂OCH₃), ethoxymethyl (—CH₂OCH₂CH₃), and methoxyethyl (—CH₂CH₂OCH₃).

The term “alkyl,” by itself or as part of another substituent, means, unless otherwise stated, a fully saturated, straight, branched chain, or cyclic hydrocarbon radical, or combination thereof, and can include di- and multi-valent radicals, having the number of carbon atoms designated (e.g., C₁-C₁₂ means from one to twelve carbon atoms, inclusive). Examples of alkyl groups include, without limitation, methyl, ethyl, n-propyl, isopropyl, n-butyl, t-butyl, isobutyl, sec-butyl, cyclohexyl, (cyclohexyl)ethyl, cyclopropylmethyl, and homologs, and isomers thereof, for example, n-pentyl, n-hexyl, n-heptyl, n-octyl, and the like. The term “alkyl,” unless otherwise noted, includes cycloalkyls.

The term “alkenyl” means an alkyl group as defined above except that it contains one or more double bonds. Examples of alkenyl groups include vinyl, 2-propenyl, crotyl, 2-isopentenyl, 2-(butadienyl), 2,4-pentadienyl, 3-(1,4-pentadienyl), etc., and higher homologs and isomers.

The term “alkynyl” means an alkyl or alkenyl group as defined above except that it contains one or more triple bonds. Examples of alkynyl groups include ethynyl, 1- and 3-propynyl, 3-butynyl, and the like, including higher homologs and isomers.

The term “aryl” refers to an aromatic hydrocarbon substituent, which may be monocyclic (e.g., phenyl), bicyclic (e.g., naphthyl, biphenyl), or polycyclic (e.g., anthracenyl, phenanthryl). The term “aryl” also encompasses “substituted aryl.” For phenyl groups, the aryl ring may be mono-, di-, tri-, tetra-, or penta-substituted. For larger aryl ring systems, the ring may be unsubstituted or substituted with one or more substituents.

The term “arylalkyl” refers to a substituent in which an alkyl group is bonded to one or more aryl groups, with the point of attachment to the parent structure occurring through the alkyl portion. The alkyl group may be linear, branched, or cyclic, and may be unsubstituted or substituted. The aryl group may be unsubstituted or substituted, and may be monocyclic, bicyclic, or polycyclic. Non-limiting examples of arylalkyl groups include benzyl (—CH₂Ph), phenethyl (—CH₂CH₂Ph), diphenylmethyl, triphenylmethyl, and naphthylmethyl.

The term “halogen” or “halo” is used herein to refer to fluorine, bromine, chlorine, and iodine atoms.

The term “heterocyclyl” refers to a saturated, partially unsaturated, or aromatic ring system containing heteroatoms independently selected from nitrogen, oxygen, or sulfur. The point of attachment to the parent structure is through a ring atom. The heterocyclyl ring may be monocyclic, bicyclic, or polycyclic, and may be unsubstituted or substituted with one or more substituents. Non-limiting examples of heterocyclyl groups include pyrrolidinyl, piperidinyl, morpholinyl, piperazinyl, and azetidinyl.

The term “hydroxy” is used herein to refer to the group —OH.

The term “methoxy” is used herein to refer to the group —OCH₃.

The term “substituted” refers to a chemical group as described herein that further includes one or more substituents, such as lower alkyl (e.g., C₁-C₁₀), aryl, acyl, halogen (e.g., alkylhalo such as CF₃), hydroxy, amino, alkoxy, alkylamino, acylamino, thioamido, acyloxy, aryloxy, aryloxyalkyl, mercapto, thia, aza, oxo, both saturated and unsaturated cyclic hydrocarbons, heterocycles and the like. These groups may be attached to any carbon or substituent of the alkyl, alkenyl, alkynyl, or other moieties described herein.

A “subject” is a living animal, particularly a mammal, which can be treated with a pharmaceutical composition described herein. In some embodiments, the subject is a human.

The terms “therapeutically effective amount” and “therapeutically effective dose” are used interchangeably herein and refer to the minimum amount of an active ingredient portion of a pharmaceutical composition required to result in a particular physiological effect (e.g., an amount required to increase, activate, enhance, decrease, or inhibit a particular physiological effect).

“Treating” as used herein with regard to a patient, refers to improving at least one symptom of the patient's disorder. Treating can be curing, improving, or at least partially ameliorating a disorder.

“Pharmaceutically suitable salt” refers to any acid or base addition salt whose counter-ions are non-toxic to the patient in pharmaceutical doses of the salts, so that the beneficial effects inherent in the free base or free acid are not vitiated by side effects ascribable to the counter-ions.

As used herein, the singular forms “a,” “an,” and “the” include plural referents unless the content clearly dictates otherwise.

As used herein, the term “or” is an inclusive “or” operator and is equivalent to the term “and/or” unless the context clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

The elements and method steps described herein can be used in any combination whether explicitly described or not.

All combinations of method steps as used herein can be performed in any order, unless otherwise specified or clearly implied to the contrary by the context in which the referenced combination is made.

The elements and methods disclosed herein can comprise, consist of, or consist essentially of the essential elements and steps described herein, as well as any additional or optional ingredients, components, or limitations described herein or otherwise useful in the art. The disclosure provided herein suitably may be practiced in the absence of any element which is not specifically disclosed herein.

It is understood that the disclosure is not confined to the particular construction and arrangement of parts herein illustrated and described, but embraces such modified forms thereof as come within the scope of the claims.

### Phytocannabinoid Derivatives

Provided here are novel phytocannabinoid derivatives intended for multimodal activity at neurological receptors implicated in neurological disorders.

In one version, referring to Scheme 1 below, CBD (1) is transformed through a sequence of four reactions to produce compound (5) which may be subsequently used as the starting material to introduce nitrogen- or oxygen-containing functionalities. The first three steps from CBD (1) to compound (4) has been described in Tchilibon and Mechoulam (“Synthesis of a Primary Metabolite of Cannabidiol,” Org. Lett. 2000, 2(21): 3301-3303) for the synthesis of 7-OH-CBD, a primary metabolite of CBD. Briefly, CBD (1) is converted into its dimethyl ether (2) which is then reacted with 3-chloroperbenzoic acid to give the epoxide (3). The subsequent ring opening of the epoxide (3) with methylmagnesium N-cyclohexylisopropyl amide in toluene leads to compound (4). Then a mesylation reaction was attempted to react compound (4) with methanesulfonyl chloride (MsCl) in the presence of triethylamine (TEA) and dichloromethane (DCM) as the solvent (Step 4 of Scheme 1). However, instead of mesylation of the hydroxy group at position 2, the reaction unexpectedly yielded compound (5) with a chlorine substitute at position 7. Using compound (5) as the starting material, compound (6) with a variety of functionalities (—R) can be obtained. The methoxy groups at positions 1′ and 5′ of compound (6) may be converted back to the hydroxy groups, forming compound (7), a homolog of CBD with functionalities at position 7. The hydroxy groups at positions 1′ and 5′ of compound (7) may be further converted into ester groups to generate compound (8), such as acetyl esters, or other functional groups that facilitate functionality of the compound.

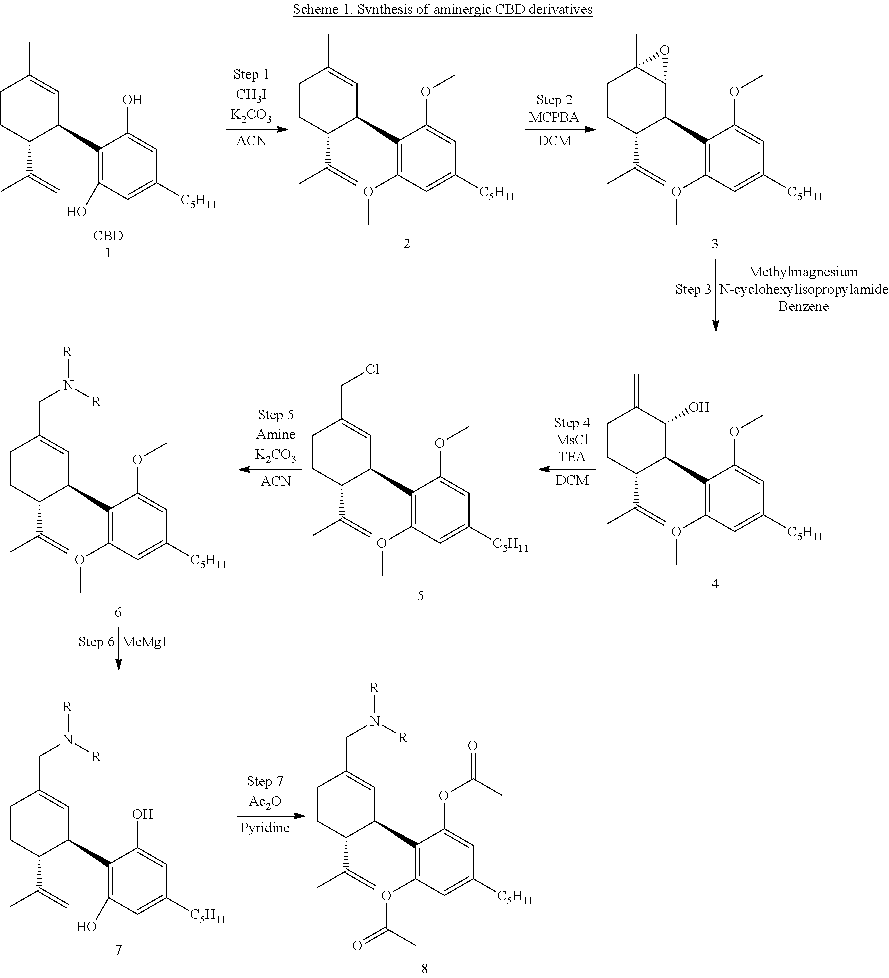

In another version, derivatives of THC, including Δ⁸-THC and Δ⁹-THC, can be obtained through a similar pathway. In an exemplary pathway shown in Scheme 2, Δ⁸-THC goes through similar reaction steps as CBD to obtain compound (Δ⁸-5) which may be subsequently used as the starting material to introduce nitrogen- or oxygen-containing functionalities. In this process, Δ⁸-THC (9) is derived from CBD (1) and firstly converted into its methyl ether (Δ⁸-2) which is then reacted with 3-chloroperbenzoic acid to give the epoxide (Δ⁸-3). Subsequent ring opening of the epoxide (Δ⁸-3) with methyl magnesium N-cyclohexylisopropyl amide in toluene leads to compound (Δ⁸-4). Then by reacting compound (Δ⁸-4) with MsCl in the presence of TEA and DCM as the solvent, compound (Δ⁸-5) with a chlorine substitute at position 11 is obtained. Using compound (Δ⁸-5) as the starting material, compound (Δ⁸-6) with a variety of functionalities (—R) can be obtained. The methoxy group at position 1 of compound (Δ⁸-6) may be converted back to the hydroxy group, forming compound (Δ⁸-7), a homolog of Δ⁸-THC with functionalities at position 11. The hydroxy group at position 1 of compound (Δ⁸-7) may be further converted into an ester group, such as an acetyl ester, or other functional groups that facilitate functionality of the compound.

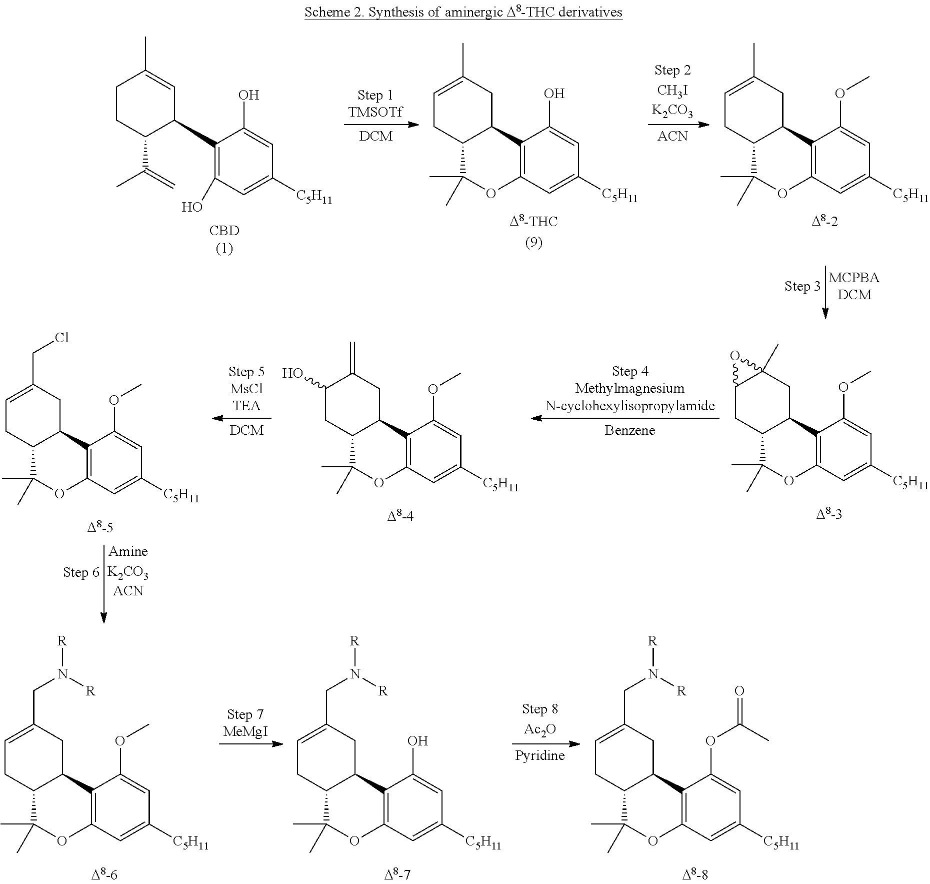

Thus, provided herein are CBD derivatives functionalized at position 7 (Formula I) and THC derivatives functionalized at position 11 (Formula II):

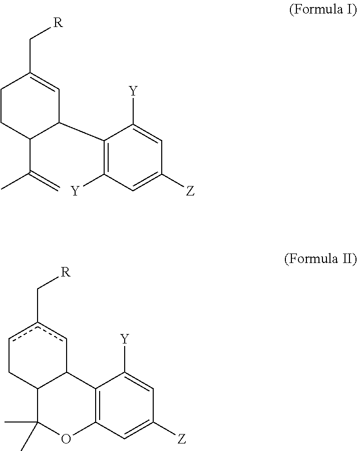

In Formulas I and II, “Y” may be a methoxy, hydroxy, ester, or acetal ester substituents. The “R” functionalities can be specifically designed targeting multiple neurological receptors. For example, compounds can be designed based on the catecholamine hypothesis of endogenous neurotransmitters in depressive disorders. CBD and Δ⁸-THC are suitable for this application as they display inherent affinity towards many relevant targets, while maintaining non- or low-psychotropic, non-toxic, and brain-penetrant properties.

In one version, the functional group “R” may be nitrogen-containing functional groups selected from:

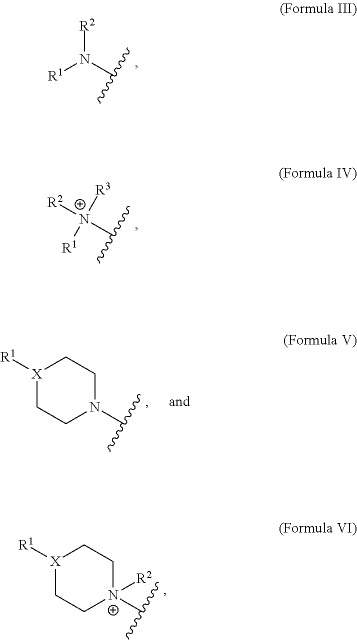

In Formulas III-VI, “R¹,” “R²,” and “R³,” if present, are each independently selected from the group consisting of hydrogen, unsubstituted or substituted C₁-C₁₂ alkyl, C₂-C₁₂ alkenyl, C₂-C₁₂ alkynyl, aryl, arylalkyl, and heterocyclyl; and “X” is selected from the group consisting of carbon, nitrogen, and oxygen.

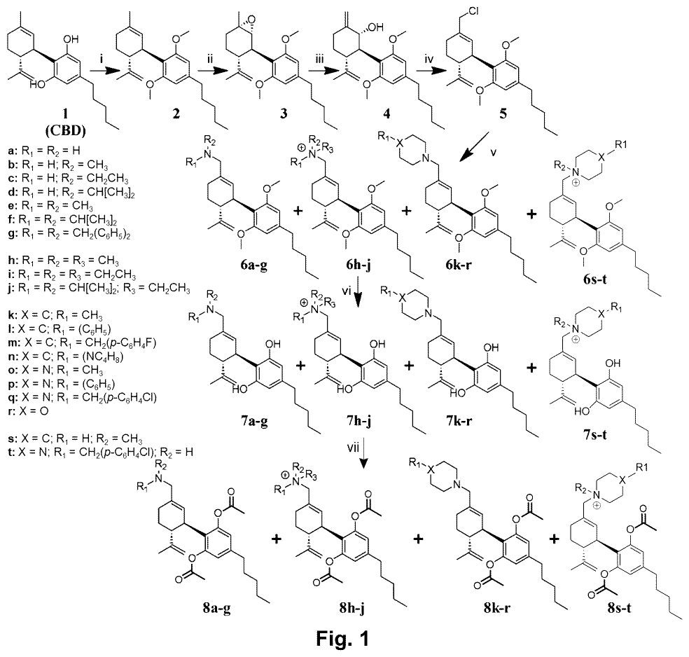

Structures of exemplary CBD derivatives 6a-6t, 7a-7t, and 8a-8t made according to the present disclosure are shown in FIG. 1. Δ⁸-THC or Δ⁹-THC derivatives containing the same functionalities can be made using similar pathways.

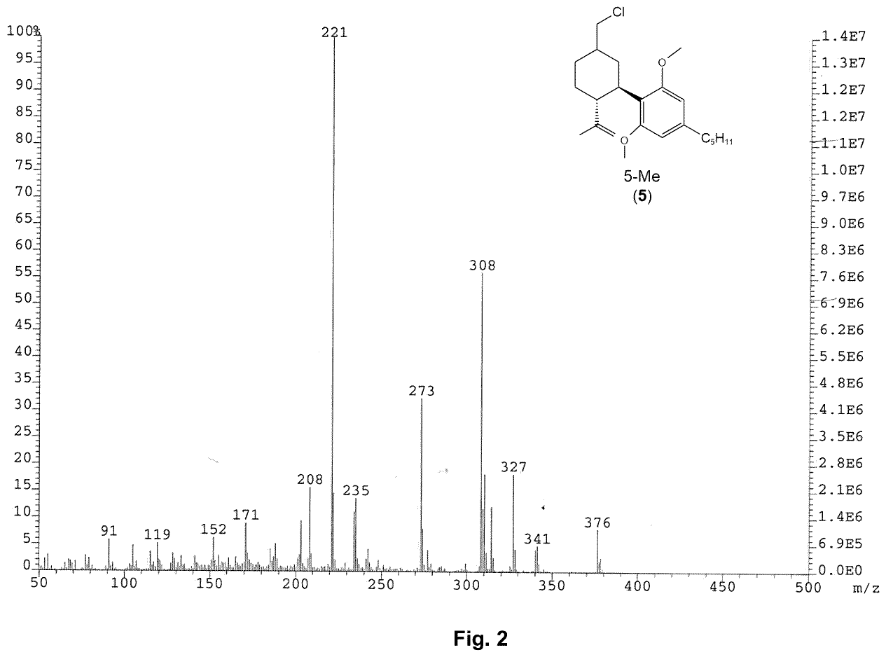

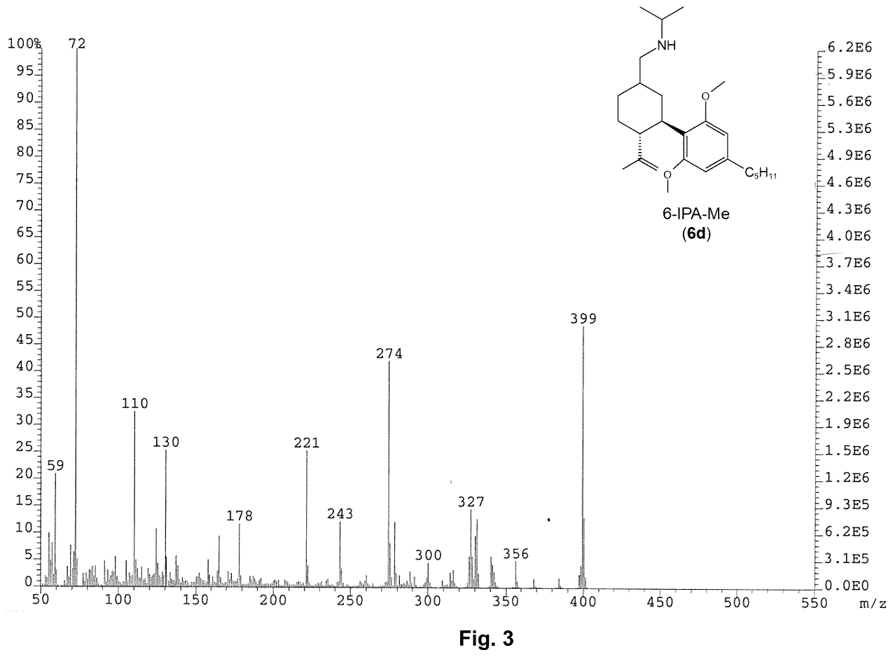

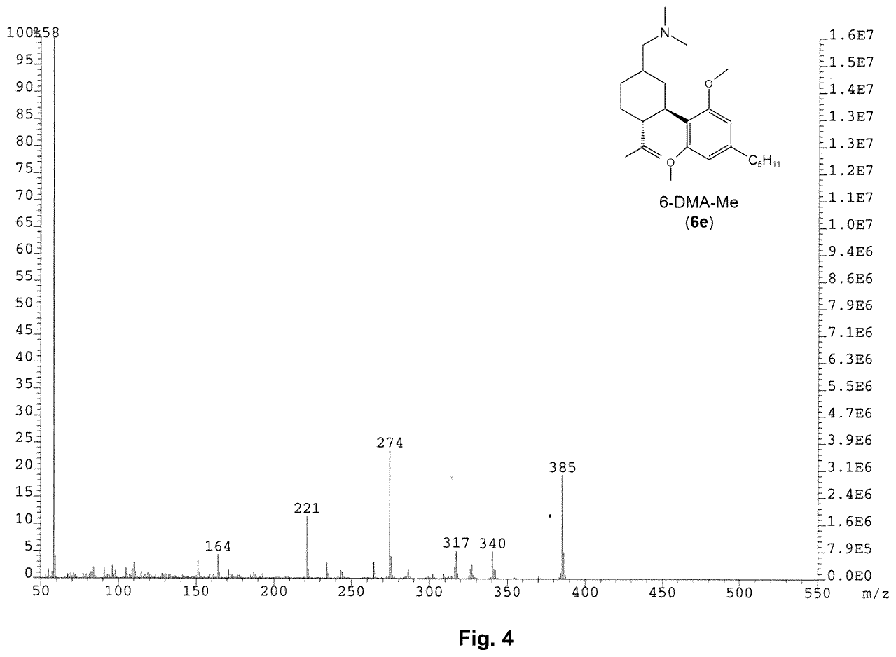

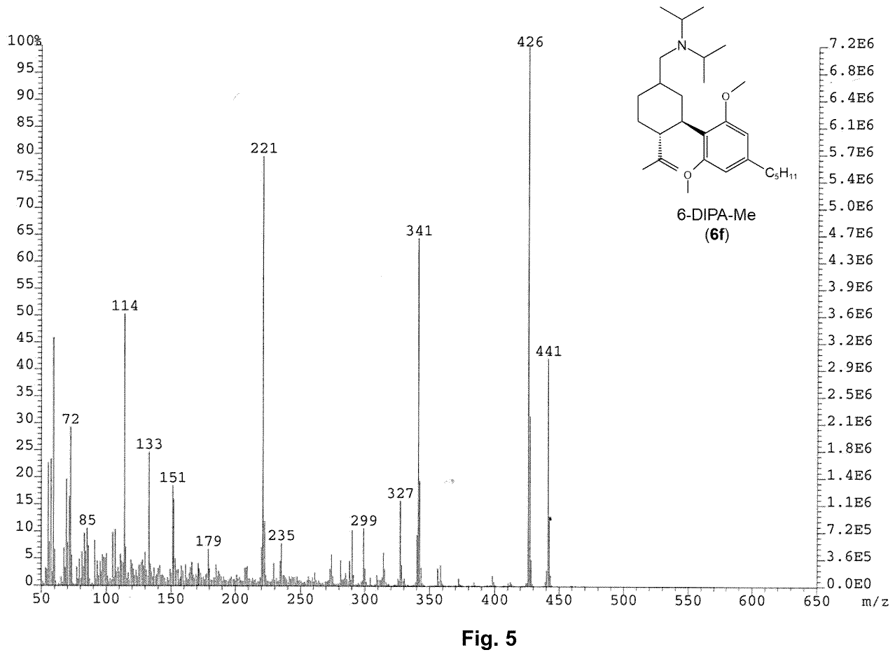

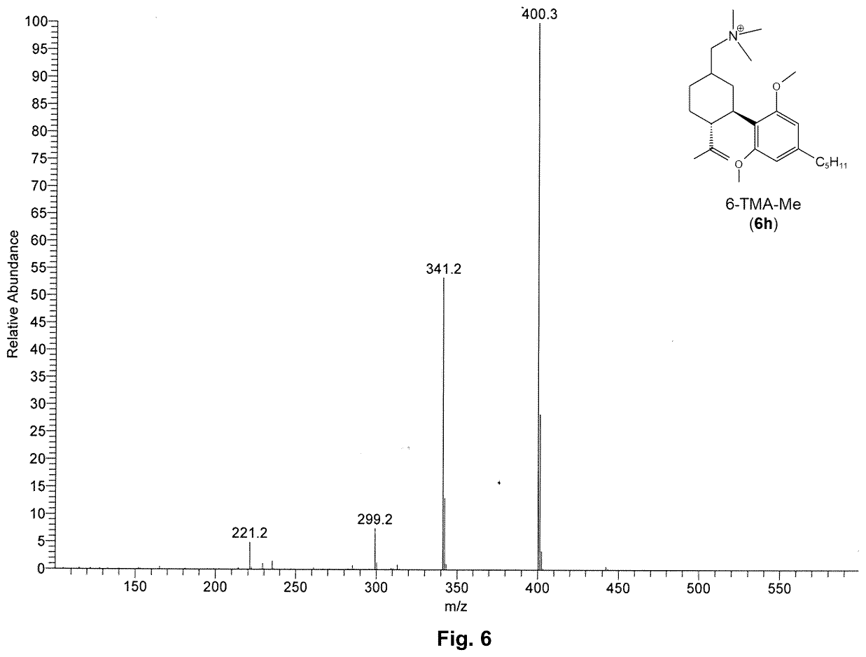

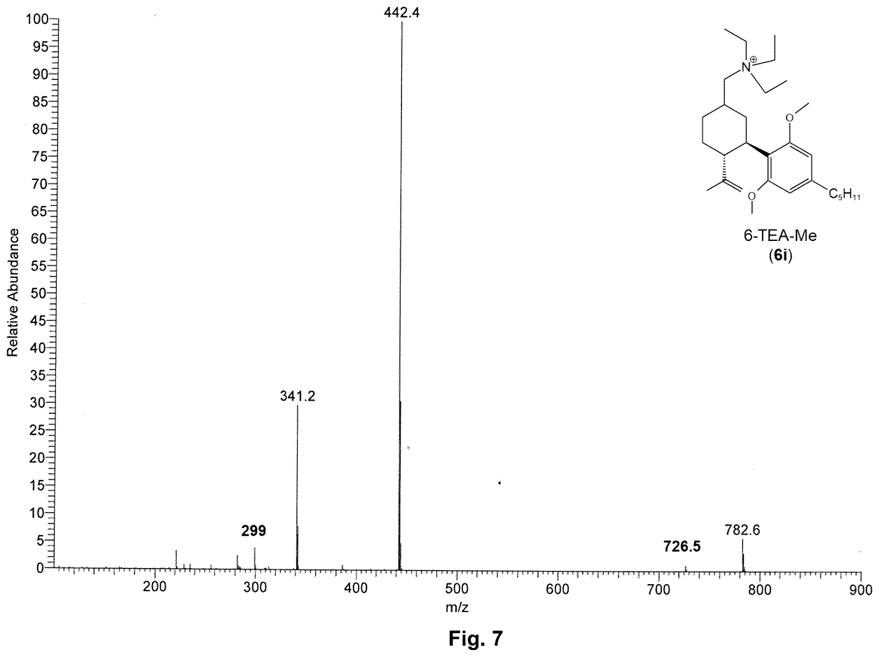

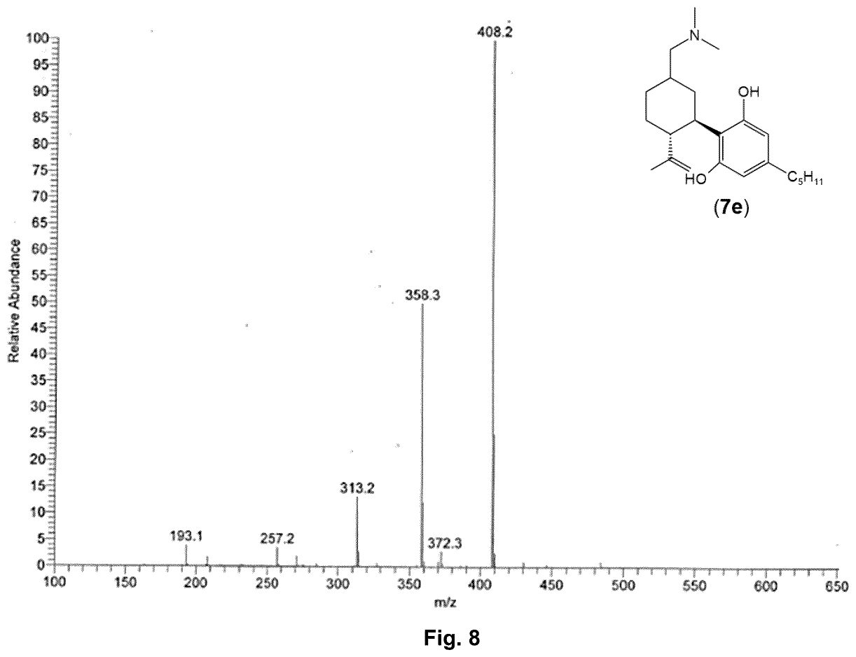

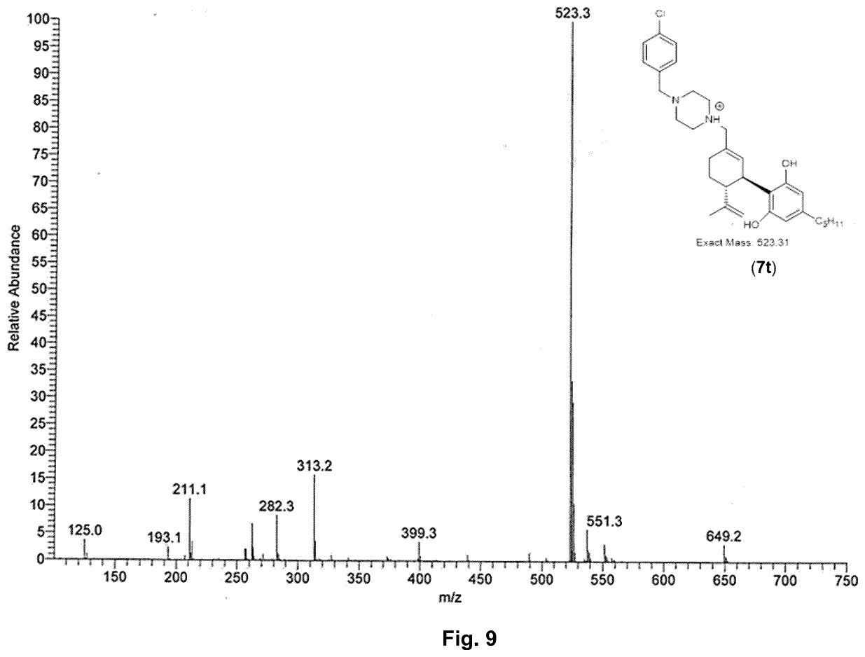

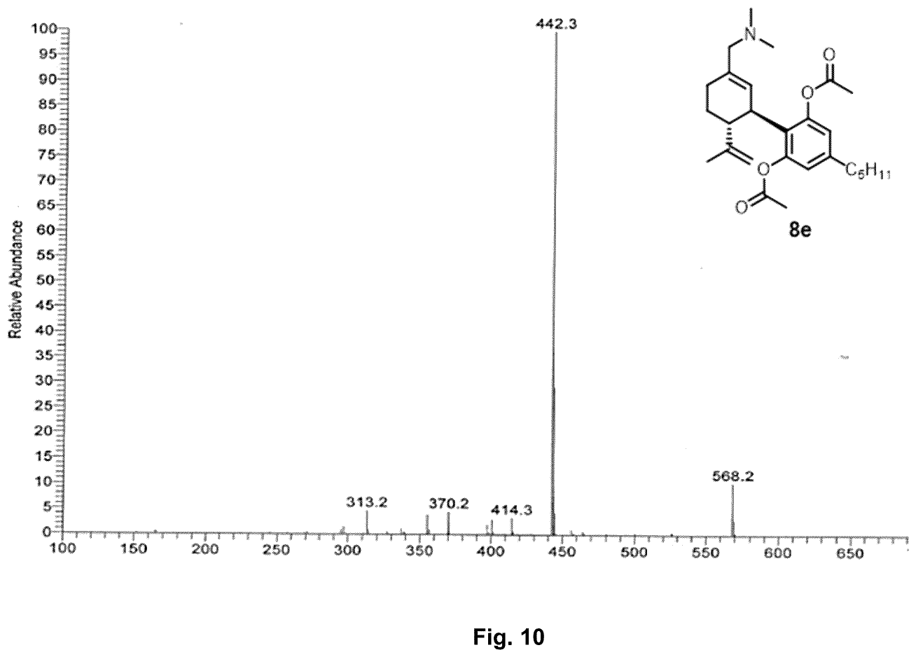

The synthetic products were subject to ¹H/¹³C-Nuclear Magnetic Resonance (NMR) Spectroscopy and Mass Spectrometry (MS) to verify the identity and purity of the products. The MS spectra of the key compound (5) of Scheme 1 and CBD derivatives 6d (6-IPA-Me), 6e (6-DMA-Me), 6f (6-DIPA-Me), 6h (6-TMA-Me), 6i (6-TEA-Me), 7e (6-DMA-OH), 7t (6-DIPA-OH), and 8e (6-DMA-Ac) are shown in FIGS. 2-10. The ¹H NMR spectroscopic data for these compounds, as well as for compounds 2, 6q (6-CBpz-Me), and 7q (7-CBpz-OH), are shown in Tables 1-3.

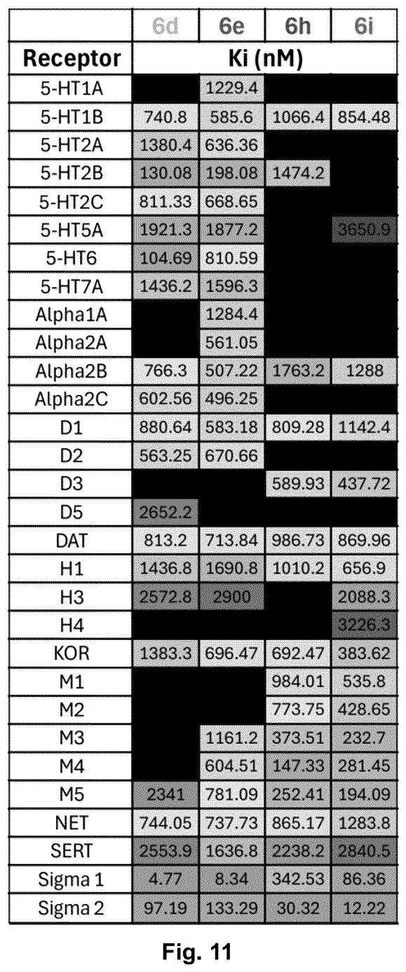

The novel amino-phytocannabinoids are comprehensively screened through the National Institute of Mental Health Psychoactive Drug Screening Program (NIMH-PDSP), which is designed to evaluate neurological target affinity and functional effects for potential central nervous system (CNS) active compounds. The compounds 6d (6-IPA-Me), 6e (6-DMA-Me), 6h (6-TMA-Me), and 6i (6-TEA-Me) were analyzed through NIMH-PDSP. The compounds first underwent primary radioligand binding assays as part of a high-throughput screening process. The assay measures the degree to which the test compounds inhibit the binding of a radiolabeled reference ligand to a specific receptor or target. It measures how effectively the compounds can block or displace the reference ligand from the target site. As shown in Table 4, each of the compounds was tested against 45 neurological receptors. Those that display >50% inhibition of the radioligand in the binding assay were subject to a binding affinity (Kᵢ) evaluation. As shown in FIG. 11, all the test compounds show strong binding affinity to Sigma 1 and Sigma 2 receptors, with lower Kᵢ values indicating higher binding affinity. Additionally, compound 6d shows strong binding affinity to 5-HT2B and 5-HT6 receptors; compound 6e shows strong binding affinity to 5HT2B receptor; compound 6h shows strong binding affinity to M4 and M5 receptors; and compound 6i shows strong binding affinity to M3, M4, and M5 receptors.

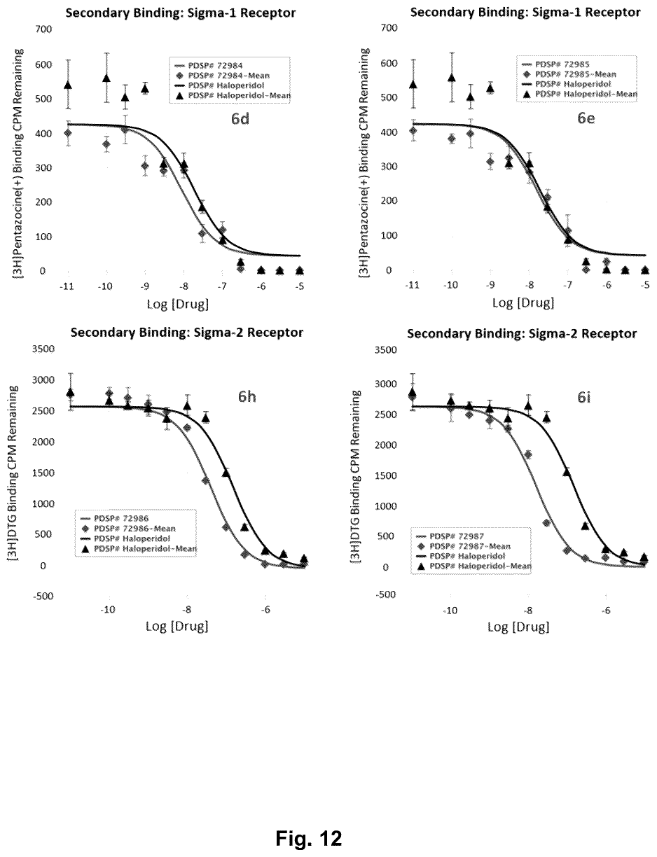

The affinity of compounds 6d and 6e to Sigma 1 receptor and compounds 6 h and 6i to Sigma 2 receptor was further compared to Haloperidol, a well-known antipsychotic medication primarily used to treat schizophrenia, acute psychosis, and Tourette syndrome. Radioligand binding assays were conducted using Pentazocine, a ligand known to bind to Sigma 1 and Sigma 2 receptors. As shown in FIG. 12, compounds 6d, 6e, 6h, and 6i exhibited higher or similar affinity to Sigma 1 and Sigma 2 receptors compared to Haloperidol.

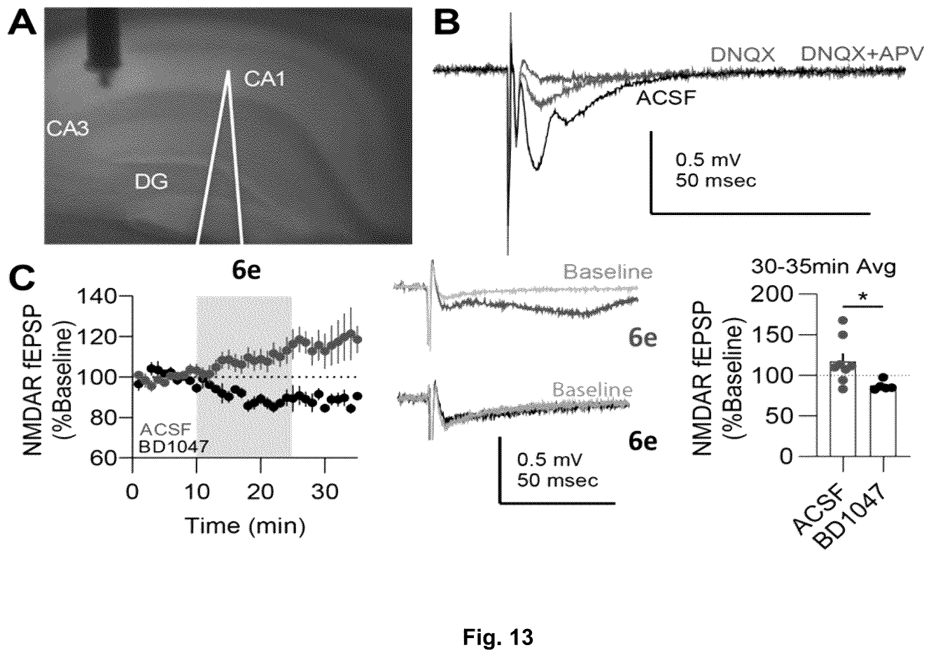

Whole-cell patch clamp experiments were utilized to evaluate the agonist/antagonist behavior of compound 6e at NMDA receptors. As shown in FIG. 13, compound 6e (6-DMA-Me) displays the ability to potentiate NMDA receptor field excitatory post-synaptic potentials (fEPSPs) in primary mouse hippocampal neurons, which can be reversed in the presence of a sigma-1 receptor antagonist, BD1047.

TABLE 1
¹H NMR spectroscopic data of selected aminergic cannabidiols measure at 400 MHz in CDCl₃ (δ_{H} mult. (J, Hz))

| | Compounds | | | |
|---|---|---|---|---|
| position | 2 | 5 | 6d | 6e |
| 2 | 1H, 5.25, s | 1H, 5.70, s | 1H, 5.40, s | 1H, 5.66, s |
| 3 | 1H, 4.03, d (8.4) | 1H, 4.08*, s br | 1H, 4.02, dd (10.5, 4.0) | 1H, 4.09-3.97, m |
| 4 | 1H, 2.94, td (10.7, 4.2) | 1H, 2.89, td (11.5, 3.1) | 1H, 2.93-2.81*, m | 1H, 2.80, ddd (13.0, 10.3, 2.8) |
| 5 | 2H, 1.86-1.75, m | 1H, 1.94-1.86, m | 1H, 1.84-1.78*, m | 1H, 1.84, dt (12.8, 3.2) |
| | | 1H, 1.80, qd (12.2, 5.2) | 1H, 1.80-1.68*, m | 1H, 1.68, qd (12.7, 5.2) |
| 6 | 1H, 2.31-2.14, m; | 1H, 2.45-2.33, m | 1H, 2.29-2.17, m | 1H, 2.49*, m |
| | 1H, 2.02, d (17.4) | 1H, 2.26, dd (16.6, 5.4) | 1H, 2.09, dd (12.3, 5.4)) | 1H, 2.26, dd (17.6, 5.3) |
| 7 | 3H, 1.70, s | 2H, 4.08*, s br | 1H, 3.18, d (15.3) | 1H, 3.44, d (12.2) |
| | | | 1H, 3.14, d (15.3) | 1H, 3.21, d (12.2) |
| 9 | 2H, 4.47 d br (6.6) | 1H, 4.51 dd (2.6, 1.4); | 1H, 4.44, d (1.5) | 1H, 4.41, s br |
| | | 1H, 4.45 d (2.8) | 1H, 4.40, d (1.5) | 1H, 4.34, s br |
| 10 | 3H, 1.64, s | 3H, 1.67*, s br | 3H, 1.60*, s br | 3H, 1.54*, s br |
| 2′ and 4′ | 2H, 6.37, s | 2H, 6.37, s | 2H, 6.32, s | 2H, 6.29, s |
| 1″ | 2H, 2.57, t (7.5) | 2H, 2.64-2.55, m | 2H, 2.53, t (7.8) | 2H, 2.49*, t (7.5) |
| 2″ | 2H, 1.68-1.60, m | 2H, 1.67*, s br | 2H, 1.60*, s br | 2H, 1.54*, s br |
| 3″ | 2H, 1.42-1.31, m | 2H, 1.44-1.34, m | 2H, 1.38-1.28, m | 2H, 1.35-1.24, m |
| 4″ | 2H, 1.42-1.31, m | 2H, 1.44-1.34, m | 2H, 1.38-1.28, m | 2H, 1.35-1.24, m |
| 5″ | 3H, 0.94, t (6.8) | 3H, 0.96, t (7.0) | 3H, 0.90, t (7.0) | 3H, 0.89-0.83, m |
| —OCH₃ | 6H, 3.77, s | 6H, 3.77, s | 6H,3.72, s | 6H, 3.69, s |
| NCH₃ | | | | 6H, 2.64, s |
| NCHCH₃ | | | 1H, 2.93-2.81*, m | |
| NCHCH₃ | | | 3H, 1.06, d (6.4) | |
| | | | 3H, 1.02, d (6.4) | |
| NCH₂CH₃ | | | | |
| NCH₂CH₃ | | | | |

| | Compounds | | |
|---|---|---|---|
| position | 6f | 6h | 6i |
| 2 | 1H, 5.38, s | 1H, 5.90, s | 1H, 5.80, s |
| 3 | 1H, 3.99 d br (10.6) | 1H, 4.05*, m | 1H, 4.00, dd (12.9, 2.4) |
| 4 | 1H, 2.86, td (15.4, 3.1) | 1H, 2.72, td (12.7, 2.8) | 1H, 2.65, ddd (13.0, 10.4, 2.8) |
| 5 | 2H, 1.81-1.66*, m | 1H, 1.81-1.74, m | 1H, 1.74, dt (12.8, 2.8) |
| | | 1H, 1.66, td, (12.2, 5.1) | 1H, 1.61, qd (12.5, 5.0) |
| 6 | 2H, 2.20-2.07*, m | 2H, 2.40-2.26*, m | 1H, 2.37-2.26, m |
| | | | 1H, 2.19, ddd (14.4, 5.0, 2.5) |
| 7 | 1H, 2.97, d (12.9) | 1H, 4.11, d (12.7) | 2H, 3.85 s br |
| | 1H, 2.92, d (12.9) | 1H, 4.01, d (12.7) | |
| 9 | 1H, 4.42, s br | 1H, 4.41, s br | 1H, 4.37, s |
| | 1H, 4.38, s br | 1H, 4.31, s br | 1H, 4.25, s |
| 10 | 3H, 1.60* s br | 3H, 1.54 s br | 3H, 1.48*, s br |
| 2′ and 4′ | 2H, 6.31, s | 2H, 6.27, s | 2H, 6.23, s |
| 1″ | 2H, 2.53, t (7.8) | 2H, 2.48, t (7.5) | 2H, 2.44, t (7.7) |
| 2″ | 2H, 1.64-1.56*, m | 2H, 1.54*, s br | 2H, 1.54-1.46*, m |
| 3″ | 2H, 1.38-1.30, m | 2H, 1.32-1.23, m | 2H, 1.25-1.19, m |
| 4″ | 2H, 1.38-1.30, m | 2H, 1.32-1.23, m | 2H, 1.25-1.19, m |
| 5″ | 3H, 0.90, t (7.0) | 3H, 0.85, t (6.8) | 3H, 0.79, t (6.8) |
| —OCH₃ | 6H, 3.71, s | 6H, 3.68, s | 6H, 3.65, s |
| NCH₃ | | 9H, 3.36, s | |
| NCHCH₃ | 2H, 3.06, p (6.6) | | |
| NCHCH₃ | 6H, 0.99, d (6.5) | | |
| | 6H, 0.93, d (6.5) | | |
| NCH₂CH₃ | | | 6H, 3.52-3.28*, m |
| NCH₂CH₃ | | | 9H, 1.30, t (7.2) |

*Overlapped

TABLE 2
¹H NMR spectroscopic data of selected aminergic cannabidiols
measure at 400 MHz in CDCl₃ (δ_{H} mult. (J, Hz))

| | Compounds | |
|---|---|---|
| position | 6q | 7q |
| 2 | 1H, 5.45, s | 1H, 5.76, s |
| 3 | 1H, 4.05, d br (10.5) | 1H, 3.94, d (10.3) |
| 4 | 1H, 2.89, ddd (13.6, 10.5, 3.0) | 1H, 2.89, td (11.5, 3.1) |
| 5 | 1H, 1.88-1.74*, m | 1H, 1.90-1.83, m |
| | 1H, 1.78-1.70*, m | 1H, 1.81-1.71*, m |
| 6 | 1H, 2.27-2.20*, m | 1H, 2.35-2.27*, m |
| | 1H, 2.10-2.19*, m | 1H, 2.25-2.18*, m |
| 7 | 1H, 2.95, d (12.1) | 1H, 2.99, d (12.6) |
| | 1H, 2.80, d (12.1) | 1H, 2.92, d (12.6) |
| 9 | 1H, 4.47, d (1.3) | 1H, 4.64, s |
| | 1H, 4.42, d (2.7) | 1H, 4.55, s |
| 10 | 3H, 1.63, s | 3H, 1.68, s br |
| 2′ and 4′ | 2H, 6.35, s | 2H, 6.19, s br |
| 1″ | 2H, 2.56, t (7.5) | 2H, 2.64-2.55, m |
| 2″ | 2H, 1.68-1.58*, m | 2H, 1.60-1.52*, m |
| 3″ | 2H, 1.42-1.30, m | 2H, 1.35-1.28, m |
| 4″ | 2H, 1.42-1.30, m | 2H, 1.35-1.28, m |
| 5″ | 3H, 0.93, t (6.9) | 3H, 0.89, t (7.0) |
| —OC{tilde under (H)}₃ | 6H, 3.73, s | |
| NC{tilde under (H)}₃ | | |
| Benzylic (Bz) | 2H, 3.52, s | 2H, 3.50, d (2.7) |
| Piperazine protons | 8H, 2.60-2.30*, m | 8H, 2.60-2.32*, m |
| Aromatic protons | 4H, 7.40-7.22*, m | 4H, 7.31-7.22*, m |

*Overlapped

TABLE 3
¹H NMR Spectroscopic data for compounds 7e and 8e
measured at 400 MHz in CDCl₃ (δ_{H} mult. (J, Hz))

| | Compound | |
|---|---|---|
| Position | 7e | 8e |
| 2 | 1H, 5.90, s | 1H, 5.39, s |
| 3 | 1H, 3.96-3.82, m | 1H, 3.56, dd (10.6, 2.1) |
| 4 | 1H, 2.79, m* | 1H, 2.67-2.58, m* |
| 5 | 1H, 1.94-1.84, m | 1H, 1.85, ddt (13.0, 5.1, 2.3) |
| | 1H, 1.73, dd (11.8, 4.8) | 1H, 1.69, tt (12.2, 6.1) |
| 6 | 1H, 2.50, t (6.0) | 1H, 2.41-2.32, m |
| | 1H, 2.31-2.20, m | 1H, 2.08-2.18, m* |
| 7 | 1H, 3.62, d (12.8) | 1H, 3.56, dd (10.6, 2.1) |
| | 1H, 3.53, d (12.6) | 1H, 2.67-2.58, m* |
| 9 | 2H, 4.55, d (16.0) | 1H, 4.56, s |
| | | 1H, 4.46, d (2.3) |
| 10 | 3H, 1.61, s | 3H, 1.63-1.51, s* |
| 2′ + 4′ | 2H, 6.30, s | 2H, 6.71, s |
| 1″ | 2H, 2.37, dd (8.9, 6.8) | 2H, 2.58-2.51, m |
| 2″ | 2H, 1.56-1.44, m | 2H, 1.63-1.51, m* |
| 3″ | 2H, 1.33-1.18, m* | 2H, 1.36-1.22, m* |
| 4″ | 2H, 1.33-1.18, m* | 2H, 1.36-1.22, m* |
| 5″ | 3H, 0.86, t (7.0) | 3H, 0.90-0.84, t (6.9) |
| —O{tilde under (H)} | Not observed | |
| —OCOOC{tilde under (H)}₃ | | 6H, 2.21, s br* |
| —NC{tilde under (H)}₃ | 6H, 2.79, s | 6H, 2.21, s br* |

*Overlapped

TABLE 4
Results of primary radioligand binding assays for compounds 6d, 6e, 6h, and 6i.

| | | | Inhibition | Inhibition | Inhibition | Inhibition | Mean |
|---|---|---|---|---|---|---|---|
| Name | Compound # | Receptor | 1 | 2 | 3 | 4 | % |
| 6-IPA-Me | 6d | 5-HT1A | 76.63 | 23.42 | 22.94 | 40.8 | 40.95 |
| | | 5-HT1B | 93.93 | 83.95 | 82.13 | 86.11 | 86.53 |
| | | 5-HT1D | 90.2 | 73.91 | 75.26 | 62.57 | 75.49 |
| | | 5-HT1E | 65.14 | 44.58 | 46.44 | 35.42 | 47.9 |
| | | 5-HT2A | 94.37 | 74.23 | 72.6 | 70.69 | 77.97 |
| | | 5-HT2B | 97.93 | 98.43 | 94.94 | 104.11 | 98.85 |
| | | 5-HT2C | 89.25 | 74.48 | 77.77 | 75 | 79.13 |
| | | 5-HT3 | −6.78 | 4.71 | 14.67 | 0.37 | 3.24 |
| | | 5-HT5A | 73.63 | 50.09 | 68.7 | 58.98 | 62.85 |
| | | 5-HT6 | 100.32 | 101.27 | 95.94 | 99.36 | 99.22 |
| | | 5-HT7A | 86.32 | 46.49 | 57.63 | 78.97 | 67.35 |
| | | Alpha1A | 73.25 | 45.39 | 40.27 | 39.12 | 49.51 |
| | | Alpha1B | 18.69 | 12.62 | 27.28 | 20.36 | 19.74 |
| | | Alpha1D | 11.33 | 2.31 | −5.08 | −7.85 | 0.18 |
| | | Alpha2A | 74.82 | 43.77 | 39.15 | 40.41 | 49.54 |
| | | Alpha2B | 90.93 | 81.3 | 80.91 | 74.97 | 82.03 |
| | | Alpha2C | 104.95 | 87.36 | 87.89 | 97.62 | 94.46 |
| | | Beta1 | 81.61 | 40.2 | 42.4 | 37.77 | 50.5 |
| | | Beta2 | 17.34 | 6.1 | 13.84 | 8.58 | 11.47 |
| | | Beta3 | 61.81 | 53.54 | 38.72 | 42.6 | 49.17 |
| | | BZP Rat | 26.34 | −28.11 | −19.71 | −24.66 | −11.54 |
| | | Brain Site | | | | | |
| | | D1 | 84.22 | 28.46 | 63.06 | 73.09 | 62.21 |
| | | D2 | 87.41 | 69.16 | 78.46 | 81.69 | 79.18 |
| | | D3 | 93.15 | 83.27 | 89.23 | 91.21 | 89.22 |
| | | D4 | 74.37 | 60.97 | 51.12 | 60.42 | 61.72 |
| | | D5 | 81.5 | 90.36 | 49.12 | 55.09 | 69.02 |
| | | DAT | 108.88 | 87.06 | 96.86 | 95.7 | 97.13 |
| | | DOR | 10.93 | −1.97 | −7.51 | 2.34 | 0.95 |
| | | H1 | 85.23 | 56.43 | 58.01 | 63.31 | 65.75 |
| | | H2 | 85.13 | 80.37 | 81.41 | 88.11 | 83.76 |
| | | H3 | 72.52 | 48.56 | 49.21 | 42.24 | 53.13 |
| | | H4 | 10.54 | −15.77 | −11.97 | −29.59 | −11.7 |
| | | KOR | 87.59 | 66.57 | 57.87 | 67.37 | 69.85 |
| | | M1 | 32.76 | −4.25 | 18.49 | 27.39 | 18.6 |
| | | M2 | −11.09 | 11.85 | 4.05 | −29.75 | −6.24 |
| | | M3 | −6.53 | 56.85 | −4.59 | −8.83 | 9.23 |
| | | M4 | 48.53 | 18.24 | 17.06 | 17.85 | 25.42 |
| | | M5 | 72.28 | 61.18 | 53.92 | 61.67 | 62.26 |
| | | MOR | 32.88 | 8.18 | −0.24 | 2.48 | 10.83 |
| | | NET | 93.78 | 90.47 | 87.12 | 93.91 | 91.32 |
| | | PBR | 1.62 | −3.04 | 6.1 | 49.68 | 13.59 |
| | | SERT | 88.05 | 40.72 | 58.51 | 58.51 | 61.45 |
| | | Sigma 1 | 96.93 | 92.49 | 90.62 | 94.81 | 93.71 |
| | | Sigma 2 | 99.08 | 94.77 | 98.19 | 103.49 | 98.88 |
| | | Alpha1A | 88.66 | 81.4 | 77.37 | 78.82 | 81.56 |
| 6-DMA-Me | 6e | 5-HT1A | 97.88 | 50.13 | 37.83 | 35.29 | 55.28 |
| | | 5-HT1B | 95.94 | 86.26 | 87.17 | 87.07 | 89.11 |
| | | 5-HT1D | 93.73 | 91.78 | 77.06 | 83.97 | 86.64 |
| | | 5-HT1E | 36.24 | 12.45 | 17.38 | 14.75 | 20.21 |
| | | 5-HT2A | 94.23 | 82.73 | 85.25 | 83.89 | 86.53 |
| | | 5-HT2B | 101.02 | 77.81 | 95.94 | 100.22 | 93.75 |
| | | 5-HT2C | 92.41 | 82.36 | 85.16 | 87.62 | 86.89 |
| | | 5-HT3 | 18.37 | 11.8 | 13.44 | 14.79 | 14.6 |
| | | 5-HT5A | 83.98 | 58.08 | 49.95 | 78.56 | 67.64 |
| | | 5-HT6 | 82.3 | 92.44 | 88.5 | 88.78 | 88.01 |
| | | 5-HT7A | 97.11 | 86.67 | 70.05 | 80.37 | 83.55 |
| | | Alpha1A | 91.97 | 52.71 | 41.32 | 38.95 | 56.24 |
| | | Alpha1B | 50.59 | 23.98 | 17.24 | 19.06 | 27.72 |
| | | Alpha1D | 7.64 | 10.41 | −4.26 | −11.03 | 0.69 |
| | | Alpha2A | 89.93 | 25.86 | 39.99 | 46.98 | 50.69 |
| | | Alpha2B | 97.13 | 76.16 | 70.75 | 72.07 | 79.03 |
| | | Alpha2C | 105.15 | 93.96 | 89.46 | 98.04 | 96.65 |
| | | Beta1 | 76.5 | 34.24 | 25.1 | 30.34 | 41.55 |
| | | Beta2 | 7.82 | 13.24 | 0.7 | 28.38 | 12.54 |
| | | Beta3 | 52.24 | 24.4 | 38.93 | 49.87 | 41.36 |
| | | BZP Rat | 0.87 | 2.17 | −38.6 | −35.41 | −17.74 |
| | | Brain Site | | | | | |
| | | D1 | 96.23 | 67.2 | 67.86 | 78.55 | 77.46 |
| | | D2 | 80.22 | 58.53 | 69.55 | 76.4 | 71.18 |
| | | D3 | 91.89 | 79.45 | 95.29 | 86.14 | 88.19 |
| | | D4 | 69.8 | 68.77 | 59.55 | 60.18 | 64.58 |
| | | D5 | 82.48 | 34.72 | 27.53 | 43.55 | 47.07 |
| | | DAT | 104.39 | 103.98 | 104.39 | 105.04 | 104.45 |
| | | DOR | 25.23 | 9.2 | 10.05 | 5.42 | 12.48 |
| | | H1 | 93.18 | 75.46 | 64.56 | 62.24 | 73.86 |
| | | H2 | 66.41 | 60.6 | 67.14 | 72.99 | 66.79 |
| | | H3 | 71.92 | 56.99 | 47.39 | 50.98 | 56.82 |
| | | H4 | 50.54 | −14.54 | −6.35 | 1.36 | 7.75 |
| | | KOR | 94.36 | 85.06 | 95.27 | 86.88 | 90.39 |
| | | M1 | 57.21 | 31.26 | 24.5 | 9.8 | 30.69 |
| | | M2 | 21.49 | 21.95 | −5.58 | −13.23 | 6.16 |
| | | M3 | 87.64 | 89.21 | 77.15 | 74.92 | 82.23 |
| | | M4 | 87.59 | 78.29 | 61.77 | 71.34 | 74.75 |
| | | M5 | 84.75 | 59.32 | 83.38 | 93.3 | 80.19 |
| | | MOR | 50.8 | 34.78 | 9.54 | 19.85 | 28.74 |
| | | NET | 99.61 | 75.63 | 78.59 | 74.02 | 81.96 |
| | | PBR | 12.23 | −9.56 | 5.01 | −7.71 | −0.01 |
| | | SERT | 85.03 | 78.78 | 78.34 | 74.57 | 79.18 |
| | | Sigma 1 | 87.98 | 90.1 | 88.18 | 91.42 | 89.42 |
| | | Sigma 2 | 94.38 | 91.68 | 97.75 | 100.79 | 96.15 |
| | | Beta2 | 7.82 | 13.24 | 0.7 | 28.38 | 12.54 |
| | | Beta2 | 7.82 | 13.24 | 0.7 | 28.38 | 12.54 |
| | | Alpha1A | 94.94 | 74.63 | 70.44 | 72.7 | 78.18 |
| 6-TMA-Me | 6h | 5-HT1A | 35.85 | 15.2 | 28.84 | 39.33 | 29.81 |
| | | 5-HT1B | 81.94 | 64.63 | 74.99 | 73.55 | 73.78 |
| | | 5-HT1D | 70.6 | 63.92 | 54.08 | 47.93 | 59.13 |
| | | 5-HT1E | 23.91 | 6.25 | 0.82 | 21 | 13 |
| | | 5-HT2A | 48.24 | 34.56 | 35.85 | 45.51 | 41.04 |
| | | 5-HT2B | 82.29 | 65.95 | 69.34 | 73.72 | 72.83 |
| | | 5-HT2C | 40.61 | 51.54 | 31.41 | 32.34 | 38.98 |
| | | 5-HT3 | 17.72 | 20.12 | 19.89 | 8.99 | 16.68 |
| | | 5-HT5A | 24.61 | 24.47 | 60.72 | 28.84 | 34.66 |
| | | 5-HT6 | 37.27 | 65.54 | 51.32 | 43.66 | 49.45 |
| | | 5-HT7A | 46.08 | 46.78 | 21.64 | 56.11 | 42.65 |
| | | Alpha1A | 50.13 | 46.4 | 36.04 | 40.3 | 43.22 |
| | | Alpha1B | 30.13 | 24.53 | 18.14 | 23.16 | 23.99 |
| | | Alpha1D | 8.97 | 3.85 | 6.31 | 10.82 | 7.49 |
| | | Alpha2A | 44.75 | 50.2 | 44.33 | 27.26 | 41.64 |
| | | Alpha2B | 85.52 | 79.59 | 56.24 | 70.62 | 72.99 |
| | | Beta1 | 26.32 | 26.69 | 25.71 | 29.37 | 27.02 |
| | | Beta2 | −7.27 | −7.83 | −10.94 | −7.04 | −8.27 |
| | | Beta3 | 47.71 | 44.4 | 47.42 | 45.34 | 46.22 |
| | | BZP Rat | 1.91 | −28.7 | −31.69 | −25.83 | −21.08 |
| | | Brain Site | | | | | |
| | | D1 | 58.04 | 63.49 | 67.97 | 52.36 | 60.47 |
| | | D2 | 39.1 | 27.31 | 39.74 | 42.04 | 37.05 |
| | | D3 | 64.14 | 56.51 | 65.6 | 66.49 | 63.19 |
| | | D4 | 22.27 | 20.93 | 27.08 | 30.94 | 25.31 |
| | | D5 | 27.63 | 28.32 | 29.7 | 33.83 | 29.87 |
| | | DAT | 90.24 | 91.51 | 82.47 | 98.33 | 90.64 |
| | | DOR | 21.75 | 16.89 | 16.27 | 6.57 | 15.37 |
| | | H1 | 79.96 | 76.4 | 81.17 | 71.57 | 77.28 |
| | | H2 | 82.04 | 64.26 | 74.56 | 77.13 | 74.5 |
| | | H3 | 43.97 | 45.23 | 41.89 | 50.11 | 45.3 |
| | | H4 | 56.54 | 19.71 | 15.68 | 23.87 | 28.95 |
| | | KOR | 85.17 | 75.97 | 90.22 | 89.01 | 85.09 |
| | | M1 | 81.67 | 69.55 | 34.26 | 57.43 | 60.73 |
| | | M2 | 78.85 | 85.89 | 77.32 | 79.31 | 80.34 |
| | | M3 | 95.01 | 96.42 | 95.3 | 95.16 | 95.47 |
| | | M4 | 99.69 | NT | 99.36 | 83.73 | 94.26 |
| | | M5 | 93.69 | 91.82 | 98.99 | NT | 94.83 |
| | | MOR | 63.01 | 49.17 | 45.37 | 65.73 | 55.82 |
| | | NET | 82.38 | 59.4 | 55.66 | 69.85 | 66.82 |
| | | PBR | 16.19 | −16.66 | 5.14 | −10.58 | −1.48 |
| | | SERT | 79.31 | 71.77 | 73.71 | 76.19 | 75.25 |
| | | Sigma 1 | 88.7 | 86.9 | 87.26 | 91.7 | 88.64 |
| | | Sigma 2 | 98.96 | 94.6 | 98.8 | 103.22 | 98.9 |
| | | Beta2 | −7.27 | −7.83 | −10.94 | −7.04 | −8.27 |
| | | Beta2 | −7.27 | −7.83 | −10.94 | −7.04 | −8.27 |
| | | Alpha1A | 70.44 | 67.22 | 61.09 | 71.09 | 67.46 |
| 6-TEA-Me | 6i | 5-HT1A | 33.91 | 41.87 | 33.53 | 44.71 | 38.51 |
| | | 5-HT1B | 87.79 | 79.06 | 81.27 | 76.28 | 81.1 |
| | | 5-HT1D | 86.82 | 75.54 | 76.1 | 74.73 | 78.3 |
| | | 5-HT1E | 16.61 | 32.68 | 9.1 | 14.26 | 18.16 |
| | | 5-HT2A | 57.22 | 37.14 | 31.22 | 34.83 | 40.1 |
| | | 5-HT2B | 56.08 | 26.09 | 36.85 | 40.14 | 39.79 |
| | | 5-HT2C | 38.12 | 40.99 | 30.19 | 32.91 | 35.55 |
| | | 5-HT3 | 21.88 | 21.77 | 20.77 | 19.77 | 21.05 |
| | | 5-HT5A | 40.86 | 54.05 | 47.87 | 72.66 | 53.86 |
| | | 5-HT6 | 45.61 | 47.6 | 61 | 32.84 | 46.76 |
| | | 5-HT7A | 35.37 | 54.55 | 49.26 | 25.48 | 41.17 |
| | | Alpha1A | 43.86 | 36.86 | 25.34 | 77.63 | 45.92 |
| | | Alpha1B | 2.55 | 4.42 | 2.5 | 6.2 | 3.92 |
| | | Alpha1D | 29.08 | 16.26 | 17.38 | 18 | 20.18 |
| | | Alpha2A | 75.66 | 15.51 | 22.08 | 28.24 | 35.37 |
| | | Alpha2B | 55.78 | 71.03 | 70.91 | 72.44 | 67.54 |
| | | Beta1 | 41.3 | 25.83 | 35.7 | 29.24 | 33.02 |
| | | Beta2 | −1.56 | 5.11 | 1.15 | 4.82 | 2.38 |
| | | Beta3 | 43.9 | 48.93 | 60.01 | 43.97 | 49.2 |
| | | BZP Rat | 5.69 | 4.65 | −16.19 | −11.83 | −4.42 |
| | | Brain Site | | | | | |
| | | D1 | 65.57 | 35.78 | 51.6 | 76.59 | 57.39 |
| | | D2 | 58.05 | 52.47 | 38.91 | 41.31 | 47.69 |
| | | D3 | 91.84 | 84.52 | 71.25 | 65.29 | 78.23 |
| | | D4 | 22.81 | 3.89 | 22.89 | 8.89 | 14.62 |
| | | D5 | 47.84 | 44.25 | 50.49 | 43.38 | 46.49 |
| | | DAT | 91.66 | 82.21 | 93.33 | 88.63 | 88.96 |
| | | DOR | 18.51 | 10.2 | 12.79 | 16.18 | 14.42 |
| | | H1 | 85.93 | 72.14 | 77.41 | 79.99 | 78.87 |
| | | H2 | 80.47 | 74.2 | 79.11 | 86.12 | 79.98 |
| | | H3 | 67.33 | 62.18 | 59.89 | 63.96 | 63.34 |
| | | H4 | 89.45 | 53.23 | 52.26 | 42.59 | 59.38 |
| | | KOR | 90.73 | 88.5 | 82.74 | 84.36 | 86.58 |
| | | M1 | 96.9 | 81.89 | 72.98 | 74.91 | 81.67 |
| | | M2 | 108.53 | 103.17 | 84.97 | 102.41 | 99.77 |
| | | M3 | 98.28 | 95.16 | 99.84 | NT | 97.76 |
| | | M4 | NT | NT | NT | 87.72 | 87.72 |
| | | M5 | NT | NT | 91.73 | 97.03 | 94.38 |
| | | MOR | 84.46 | 47.54 | 39.67 | 46.18 | 54.46 |
| | | NET | 87.03 | 69.54 | 67.15 | 72.07 | 73.95 |
| | | PBR | 17.09 | 6.16 | −4.32 | 14.28 | 8.3 |
| | | SERT | 77.37 | 54.63 | 53.76 | 64.98 | 62.69 |
| | | Sigma 1 | 91.26 | 94.37 | 97.53 | 95.89 | 94.76 |
| | | Sigma 2 | 105.76 | 105.92 | 95.93 | 96.92 | 101.13 |
| | | Beta2 | −1.56 | 5.11 | 1.15 | 4.82 | 2.38 |
| | | Beta2 | −1.56 | 5.11 | 1.15 | 4.82 | 2.38 |
| | | Alpha1A | 61.74 | 56.58 | 51.9 | 69.15 | 59.84 |

“NT” = Not Tested.

### Pharmaceutical Compositions

Also disclosed herein are pharmaceutical compositions comprising one or more of the aminergic phytocannabinoid derivatives or a pharmaceutically suitable salt thereof as described herein. More specifically, the pharmaceutical composition may comprise one or more of the CBD and THC derivatives as well as a standard, well-known, non-toxic pharmaceutically suitable carrier, adjuvant or vehicle such as, for example, phosphate buffered saline, water, ethanol, polyols, vegetable oils, a wetting agent or an emulsion such as a water/oil emulsion. The composition may be in either a liquid, solid or semi-solid form. For example, the composition may be in the form of a tablet, capsule, ingestible liquid or powder, injectable, suppository, or topical ointment or cream. Proper fluidity can be maintained, for example, by maintaining appropriate particle size in the case of dispersions and by the use of surfactants. It may also be desirable to include isotonic agents, for example, sugars, sodium chloride, and the like. Besides such inert diluents, the composition may also include adjuvants, such as wetting agents, emulsifying and suspending agents, sweetening agents, flavoring agents, perfuming agents, and the like.

Suspensions, in addition to the active compounds, may comprise suspending agents such as, for example, ethoxylated isostearyl alcohols, polyoxyethylene sorbitol and sorbitan esters, microcrystalline cellulose, aluminum metahydroxide, bentonite, agar-agar and tragacanth or mixtures of these substances.

Solid dosage forms such as tablets and capsules can be prepared using techniques well known in the art of pharmacy. For example, the phytocannabinoid derivatives produced as described herein can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant phytocannabinoid derivative.

For intravenous, intramuscular, and subcutaneous administration, the phytocannabinoid derivatives may be incorporated into commercial formulations such as Intralipid©-brand fat emulsions for injection. (“Intralipid” is a registered trademark of Fresenius Kabi AB, Uppsalla, Sweden.) Where desired, the individual components of the formulations may be provided individually, in kit form, for single or multiple doses.

Possible routes of administration of the pharmaceutical compositions include, for example, enteral (e.g., oral and rectal) and parenteral. For example, a liquid preparation may be administered, for example, orally or rectally. Additionally, a homogenous mixture can be completely dispersed in water, mixed under sterile conditions with physiologically acceptable diluents, preservatives, buffers or propellants in order to form a spray or inhalant. The route of administration will, of course, depend upon the desired effect and the medical state of the subject being treated. The dosage of the composition to be administered to the patient may be determined by one of ordinary skill in the art and depends upon various factors such as weight of the patient, age of the patient, immune status of the patient, etc., and is ultimately at the discretion of the medical professional administering the treatment.

With respect to form, the composition may be, for example, a solution, a dispersion, a suspension, an emulsion, or a sterile powder which is then reconstituted. The composition may be administered in a single daily or weekly dose or multiple daily or weekly doses.

The present disclosure also includes treating neurological disorders in mammals, including humans, by administering a therapeutically effective amount of one or more the aminergic phytocannabinoid derivatives described herein. In particular, the compositions of the present disclosure may be used to treat neurological disorders of any and all description.

The above-described pharmaceutical compositions may be utilized in connection with non-human animals, both domestic and non-domestic, as well as humans.

### EXAMPLES

### Conversion of Compound 4 (4-Me) to Compound 5 (5-Me)

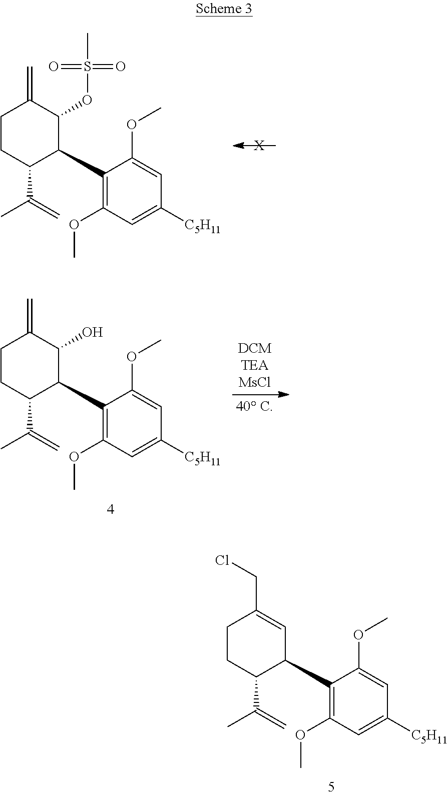

0.1964 g compound 4 was transferred to a flask, then dissolved in 3.2 mL anhydrous DCM. After purging with N₂, 155 μL TEA was added. After about 10 min, 51 μL MsCl was added. The reaction was then heated to 40° C. for 105 min. Solvent was removed under reduced pressure and the residue was reconstituted in a mixture of hexanes and H₂O. The reaction mixture was then transferred to a separatory funnel and the organic phase was collected and dried over anhydrous sodium sulfate. The salt was filtered off, and the crude product was dried under reduced pressure and then directly purified by MPLC (99% hexanes:1% ethyl acetate). Instead of mesylation of the hydroxy group at position 2 of compound 4, the reaction unexpectedly yielded compound 5 directly with a chlorine substitute at position 7.

### Conversion of Compound 5 (5-Me) to Compound 6d (6-IPA-Me)

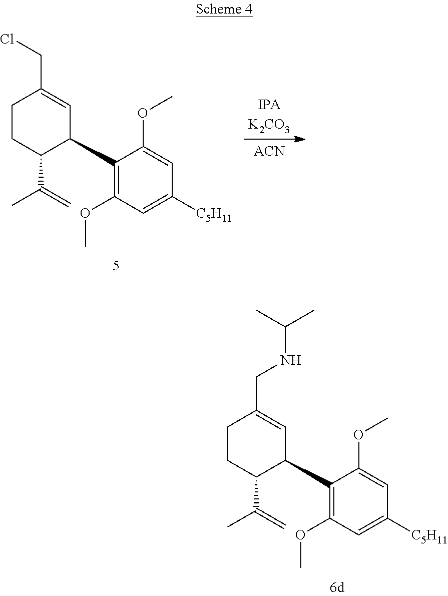

0.0444 g 5-Me was dissolved in 5 mL ACN, and purged with N₂. While purging, 163 mg K₂CO₃ and 51 μL IPA were added to the reaction, which was then heated to 40° C. and stirred overnight (18 hours). Excess IPA was used in the reaction to prevent dimerization. Solvent was removed under reduced pressure and the residue was reconstituted in a mixture of DCM:H₂O. The reaction mixture was then transferred to a separatory funnel and the organic phase was collected. The aqueous phase was re-extracted twice with DCM, and the combined organic extracts were dried over anhydrous sodium sulfate. The salt was filtered off, and the crude product was dried under reduced pressure and then directly purified by MPLC (25% DCM:75% MeOH).

### Conversion of Compound 5 (5-Me) to Compound 6e (6-DMA-Me)

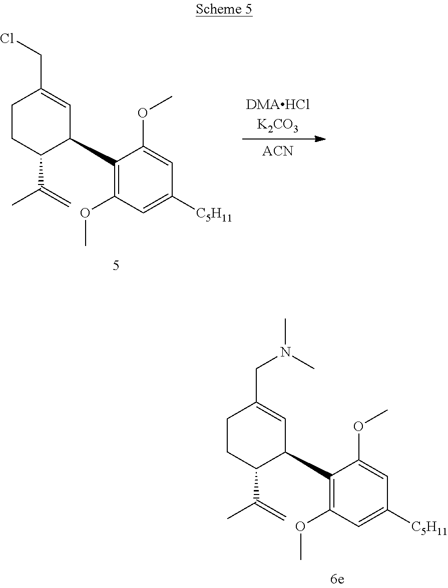

47.5 mg 5-Me was dissolved in 2 mL ACN, and purged with N₂. Then 88 mg K₂CO₃ was added while purging. The reaction was sealed and protected with N₂ balloon, and then 21 mg DMA·HCl was added to the reaction. The reaction was heated to 40° C. and stirred overnight (18 hours).

### Conversion of Compound 5 (5-Me) to Compound 6f (6-DIPA-Me)

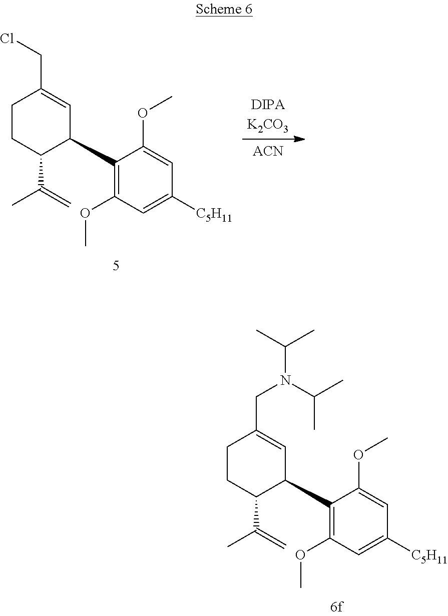

4 mL of ACN was transferred to a flask and purged with N₂. Then 83.6 mg K₂CO₃ and 83 μL DIPA were added under purging. 44.6 mg compound 5 was dissolved in 1 mL ACN and then was added dropwise to the reaction. The reaction was heated to 40° C. and stirred overnight (18 hours).

### Conversion of Compound 5 (5-Me) to Compound 6f (6-DIPA-Me), Alternative Method

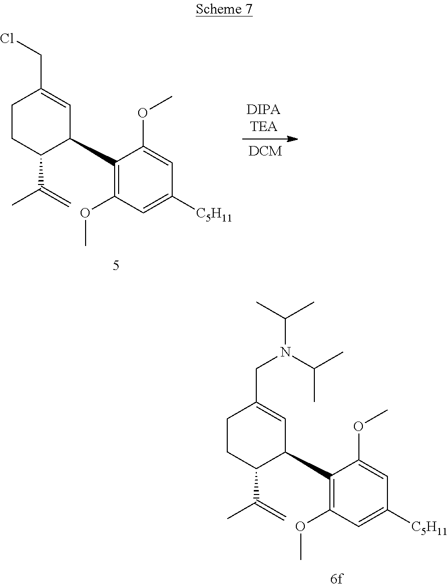

5 mL of DCM was transferred to a flask and purged with N₂. Then 85 μL TEA and 102 μL DIPA were added. The reaction was heated to 40° C. and 0.1096 g compound 5 was dissolved in 1 mL DCM then added dropwise to the reaction. The reaction was stirred overnight (18 hours).

### Conversion of Compound 5 (5-Me) to Compound 6h (6-TMA-Me)

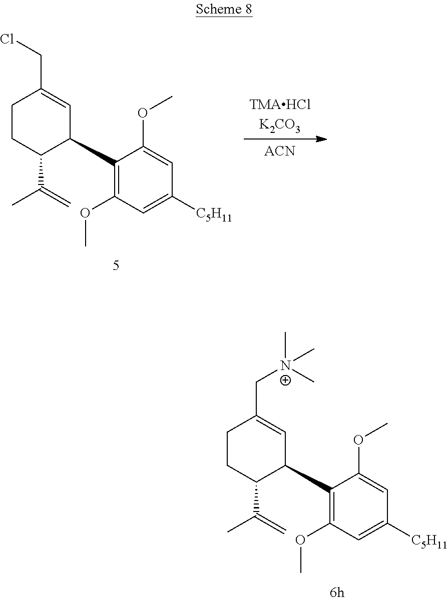

0.0677 g 5-Me was dissolved in 5 mL ACN, and purged with N₂. Then 0.1243 g K₂CO₃ and 0.0344 g TMA·HCl were added to the reaction. The reaction was heated to 40° C. and stirred overnight (18 hours).

### Conversion of Compound 5 (5-Me) to Compound 6i (6-TEA-Me)

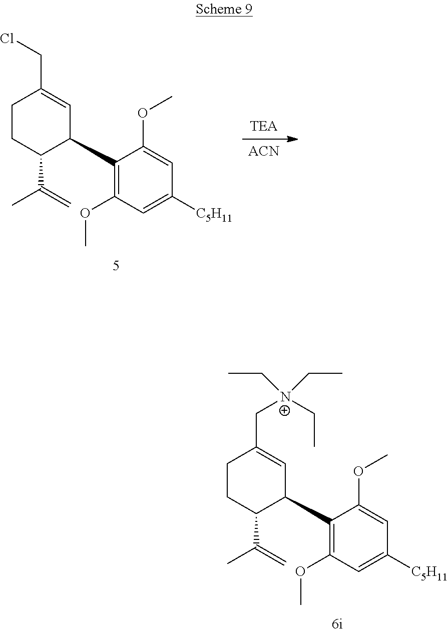

0.2327 g 5-Me was dissolved in 10 mL ACN, and purged with N₂. Then 200 μL TEA was added and the reaction was refluxed overnight at 82° C.

### Conversion of Compound 5 (5-Me) to Compound 6q (6-CBpz-Me)

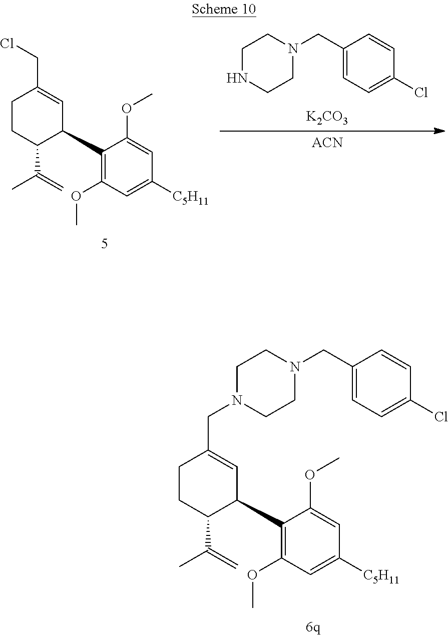

0.1048 g 5-Me was dissolved in 5 mL ACN, and purged with N₂. Added 185 mg K₂CO₃ while purging, sealed, and then added 0.102 mL 1-(4-chlorobenzyl)piperazine. The reaction was heated to 40° C. and stirred overnight.

### Conversion of Compound 6e (6-DMA-Me) to Compound 7e (7-DMA-OH)

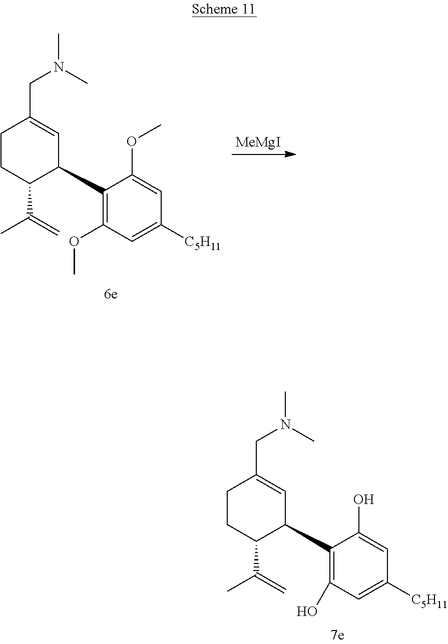

0.106 g compound 6e was placed in a small glass vial, then 2 mL of MeMgI (3.0M in diethyl ether) was added to the vial and the solvent was carefully removed under vacuum. The reaction residue was then heated to 160° C. for 1 hr. The residue was cooled to room temperature, dissolved in diethyl ether, and quenched slowly with water then 1.0 M HCl. The reaction mixture was then transferred to a separatory funnel and the organic phase was collected. The aqueous phase was re-extracted twice with ethyl acetate, and the organic phases were combined and dried over anhydrous sodium sulfate. The salt was filtered and the solvent was removed under reduced pressure. The residue was purified directly by MPLC (50% DCM: 50% MeOH).

### Conversion of Compound 6q (6-CBpz-Me) to Compound 7q (7-CBpz-OH)

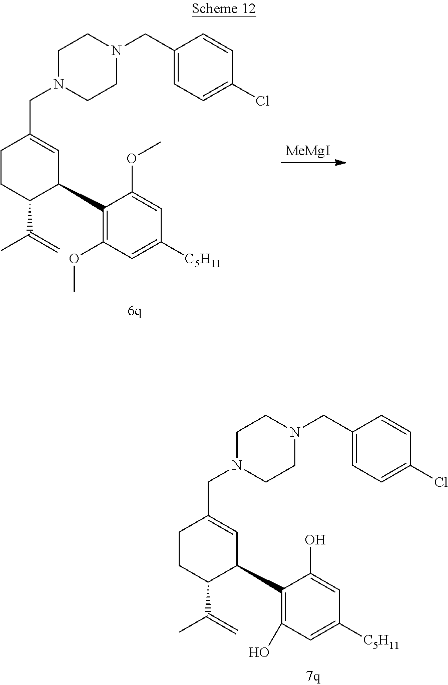

0.0627 g compound 6q was placed in a small glass vial, then 2 mL of MeMgI (3.0M in diethyl ether) was added to the vial and the solvent was carefully removed under vacuum. The reaction residue was then heated to 160° C. for 1 hr. The residue was cooled to room temperature, dissolved in diethyl ether, and quenched slowly with water then 1.0 M HCl.

### Conversion of Compound 7e (6-DMA-Me) to Compound 8e (7-DMA-Ac)

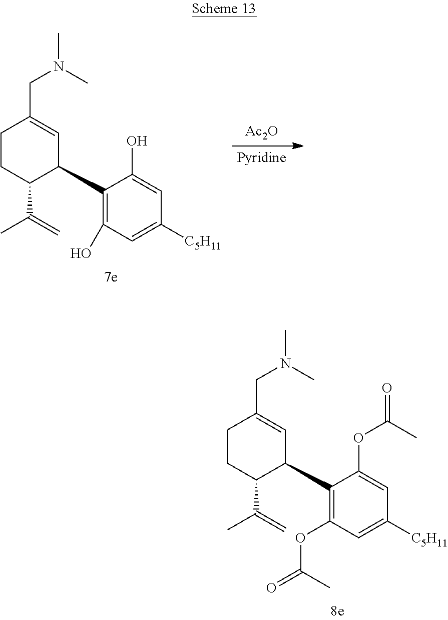

0.0531 g compound 7e was thoroughly dried in a small vial, then dissolved in 1 mL pyridine. 1 mL of Ac₂O was then added to the vial, and the reaction was stirred overnight at room temperature. The reaction mixture was then poured over a mixture of diethyl ether and water then transferred to a separatory funnel. The organic phase was collected and the aqueous phase was re-extracted twice with ethyl acetate, and the organic phases were combined and dried over anhydrous sodium sulfate. The salt was filtered and the solvent was removed under reduced pressure, and the resulting residue was purified by MPLC (DCM:MeOH).

What is claimed is:

1. A compound of Formula I or II: 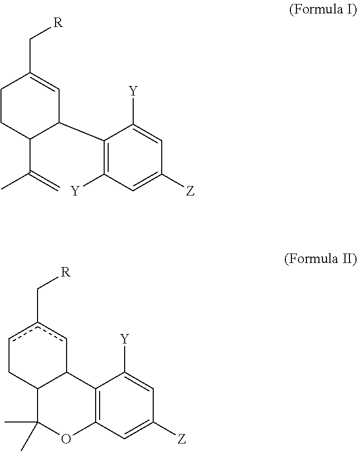
wherein each --- between adjacent atoms in Formula II represents a bond that is present or absent, provided that one bond is present and another is absent;
Y in each Formula is independently selected from the group consisting of hydroxy, alkoxy, acyloxy, and —COOR⁰, wherein R⁰ is selected from the group consisting of unsubstituted or substituted C₁-C₁₂ alkyl, C₂-C₁₂ alkenyl, C₂-C₁₂ alkynyl, and alkoxyalkyl;
Z in each Formula is independently selected from the group consisting of C₁-C₁₂ alkyl; and
each R is independently selected from the group consisting of 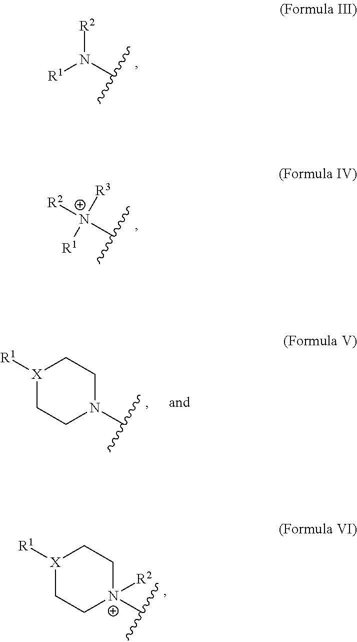
wherein R¹, R², and R³, if present, are each independently selected from the group consisting of hydrogen, unsubstituted or substituted C₁-C₁₂ alkyl, C₂-C₁₂ alkenyl, C₂-C₁₂ alkynyl, aryl, arylalkyl, and heterocyclyl; and
X is selected from the group consisting of carbon, nitrogen, and oxygen.

2. The compound of claim 1, wherein the compound is Formula I, and Y is methoxy.

3. The compound of claim 1, wherein the compound is Formula I, and Y is hydroxy.

4. The compound of claim 1, wherein the compound is Formula I, and Y is acetoxy.

5. The compound of claim 1, wherein the compound is Formula II, and Y is methoxy.

6. The compound of claim 1, wherein the compound is Formula II, and Y is hydroxy.

7. The compound of claim 1, wherein the compound is Formula II, and Y is acetoxy.

8. The compound of claim 1, wherein R is Formula III.

9. The compound of claim 8, wherein R¹ and R² are each independently selected from the group consisting of hydrogen, unsubstituted or substituted C₁-C₁₂ alkyl, and arylalkyl.

10. The compound of claim 1, wherein R is Formula IV.

11. The compound of claim 10, wherein R¹, R², and R³ are each independently selected from the group consisting of unsubstituted or substituted C₁-C₁₂ alkyl.

12. The compound of claim 1, wherein R is Formula V.

13. The compound of claim 12, wherein R¹ is selected from the group consisting of unsubstituted or substituted C₁-C₁₂ alkyl, aryl, arylalkyl, and heterocyclyl.

14. The compound of claim 13, wherein the arylalkyl is a halogen substituted arylalkyl.

15. The compound of claim 1, wherein R is Formula VI.

16. The compound of claim 15, wherein R¹ and R² are each independently selected from the group consisting of hydrogen, unsubstituted or substituted C₁-C₁₂ alkyl, and arylalkyl.

17. The compound of claim 16, wherein the arylalkyl is a halogen substituted arylalkyl.

18. A pharmaceutical composition comprising a compound as recited in claim 1, optionally in combination with a pharmaceutically suitable carrier.

19. A method of treating neurological disorders in a mammal, comprising administering a therapeutically effective amount of a compound as recited in claim 1.

20. The method of claim 19, wherein the mammal is a human.
